(19) United States
(12) Patent Application Publication
(10) Pub. No.: US 2005/0013776 A1
(43) Pub. Date: Jan. 20, 2005

(54) Methods of evaluating the dynamics of caloric restriction and identifying caloric restriction mimetics

(76) Inventors: Stephen R. Spindler, Riverside, CA (US); Joseph M. Dhahbi, Riverside, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/622,160

(22) Filed: Jul. 16, 2003

(51) Int. Cl. C12Q 1/68; A61K 49/00

(52) U.S. Cl. 424/9.1; 435/6

(57) ABSTRACT

A method of evaluating the dynamics of caloric restriction (CR). In one embodiment, a method of evaluating the dynamics of CR comprises obtaining control data from an administration of a long-term control diet program. Each of several mammalian sample groups is subjected to a CR diet program for a different amount of time. The effect of CR between each of the several mammalian sample groups and the control data are compared to each other. Additionally, the effects among members of CR among the several mammalian sample groups are compared to each other.

### BACKGROUND

1. Field

Many aspects of this disclosure relate to methods of evaluating dynamics of caloric restriction (CR) which may be used in identifying a CR mimetic.

2. Discussion of Related Art

A major goal of pharmaceutical research has been to discover ways to reduce morbidity and delay mortality. Several decades ago it was discovered that a decrease in caloric intake, termed caloric restriction, can significantly and persistently extend healthy life in animals; see for example, Weindruch, et al., The Retardation of Aging and Disease by Dietary Restriction, (Charles C. Thomas, Springfield, Ill.) 1988. CR remains the only reliable intervention capable of consistently extending lifespan and reducing the incidence and severity of many age-related diseases, including cancer, diabetes, and cardiovascular disease. Additionally, physiological biomarkers linked to lifespan extension in rodents (e.g., mice, shrews, and squirrels), other mammals (e.g., rabbits) and monkeys that have been subjected to CR have been shown to be associated with extended lifespan in humans; see for examples, Weyer, et al., Energy Metabolism after Two Years of Energy Restriction: the Biosphere Two Experiment, Am. J. Clin. Nutr. 72, 946-953, 2000, and Roth, et al., Biomarkers of Caloric Restriction may Predict Longevity in Humans, Science 297, 811, 2002. A study by Walford et al. indicated that healthy nonobese humans on CR diet programs show physiologic, hematologic, hormonal, and biochemical changes resembling those of rodents and monkeys on such CR diets. See Walford, et al., Calorie Restriction in Biosphere Two: Automations in Physiologic, Hematologic, Hormonal and Biochemical Parameters in Humans Restricted for a Two-Year Period, J. Gerontol.: Biol. Sci. 57A, 211-224, 2002. These preliminary findings suggest that the anti-aging effects of CR may be universal among all species. The molecular and genetic processes that lead to lifespan extension in animals may extend lifespan in humans.

Most CR studies have led to the widespread idea that CR acts incrementally to prevent the age-related accumulation of deleterious or harmful changes in biological macromolecules and in gene expression. This idea caused many investigators to undervalue the effects of CR on the genes that do not change in expression with age. Additionally, the detailed dynamics or kinetics of the transition of the CR phenotype remain unclear in these studies. Understanding the dynamics of CR enables effective use of CR to perform various treatments, for instance, extending longevity or delaying the onset of age-related diseases. Understanding the dynamics of CR also enables the discovery of CR mimetics that can be used efficiently to treat diseases of animals and humans.

### SUMMARY OF DISCLOSURE

In some embodiments, methods of evaluating the initial effect of CR on longevity and gene expression are disclosed. The results obtained from these embodiments indicate that the effects of CR on lifespan are induced rapidly after a shift from the normal diet to the restricted diet (e.g., CR diet program). They also indicate that the gene expression effects are rapidly induced in a stepwise manner. In addition the gene expression effects of CR are rapidly reversible. The results from these embodiments have major implications for fully understanding CR and CR dynamics.

In one exemplary embodiment, a method of evaluating the dynamics of CR is disclosed. The method comprises obtaining control data from administering a long-term control diet program. Next, each of several mammalian sample groups is subjected to a CR diet program for a different amount of time relative to other sample groups. The effects of CR between each of the several mammalian sample groups and the control data are compared to each other. Additionally, the effects of CR for different amounts of time are analyzed.

In another exemplary embodiment, a method of evaluating the dynamics of CR is disclosed. The method comprises dividing a mammalian sample group into a first sample group and second sample group. The first sample group is subjected to a long-term control diet program (e.g., a normal, non CR diet) for a first predetermined period. The second sample group is subjected to a long-term caloric restriction diet program for a second predetermined period. After the first predetermined period, portions of the first sample group are switched to a caloric restriction diet program for different amounts of time. After the second predetermined period, at least a portion of the second sample group is switched to a control diet program for a third predetermined period and the remaining portion of the second sample group is maintained on the long-term caloric restriction diet program. Effects of CR among members of the first sample group and the second sample group are compared to one another.

In another exemplary embodiment, a method of reversing some effects of CR is disclosed. The method comprises administering a control diet program to a mammalian sample group that has been subjected to a long-term caloric restriction diet program, wherein the control diet program includes higher caloric intake for the mammalian sample group than the caloric intake for a long-term caloric restriction diet program.

In another exemplary embodiment, a method of extending longevity in an old mammal is disclosed. The method comprises administering a caloric restriction diet program to the old mammal. In one case the old mammal is an old mouse. The old mouse may be more than 18 months old. In another case, the old mammal is a human of about more than 50 years old. Additionally, administering the CR diet program includes shifting the old mammal to the CR diet program in stages, with at least one stage including a gradual decrease in the number of calories in the diet program.

In another exemplary embodiment, a method of identifying an intervention for use in old subjects is disclosed. The method comprises administering a control diet program (e.g., a diet with a normal amount of calories) to individuals in the first sample group. After start of old age, at least one candidate intervention is administered to the individuals in the first sample group. The effects of the candidate intervention are compared to the effects from a CR or control or another diet program administered to the second sample group. Normally, a single candidate intervention may be administered to individuals in the first sample group in order to avoid interactions between interventions. However, it is also desirable to perform alternative methods in which a group of two or more candidate interventions is administered concurrently to individuals in another first sample group to observe the effects from the group of interventions.

In another exemplary embodiment, a method of identifying an intervention and performing at least one biochemical measurement after exposing a biological sample to the intervention is disclosed. The biochemical measurement is designed to show whether the intervention mimics substantially or at least some of the effects of CR. The intervention is then withdrawn from the biological sample. At least one further biochemical measurement is performed after withdrawing the intervention. At least one further biochemical measurement is made to determine whether withdrawing the intervention mimics substantially or at least some of the effects of withdrawing CR. Normally, a single candidate intervention may be administered to a biological sample in order to avoid interactions between interventions. However, it is also desirable to perform alternative methods in which a group of two or more candidate interventions is administered concurrently to another biological sample to observe the effects from the group of interventions.

These and other features and advantages of embodiments of the present invention will be more readily apparent from a detailed description of the embodiments, set forth below, taken in conjunction with the accompanying drawings.

### BRIEF DESCRIPTION OF THE DRAWINGS

Exemplary embodiments of the present invention are illustrated by way of example and not limitation in the figures of the accompanying drawings, in which like references indicate similar elements and in which:

FIG. 1 illustrates an exemplary embodiment in which various diet programs (for different periods of time) are administered to old mammals such as old mice;

FIG. 2 illustrates the effect of CR on longevity of mice that are subjected to CR at an old age;

FIG. 3 illustrates diet programs that are administered to mice in accordance with some embodiments of the present invention;

FIGS. 4A-4B illustrate the dynamics of changes in expression of genes whose expression is affected by CR;

FIGS. 5A-5E illustrate a result using real-time reverse transcriptase PCR (real time RT-PCR) to validate the changes in gene expression of the genes affected by CR; PCR is Polymerase Chain Reaction;

FIG. 6 illustrates a method of identifying an intervention in accordance with some embodiments of the present invention;

FIG. 7 illustrates a method of identifying an intervention for use in mammalian subjects of old age;

FIG. 8 illustrates an exemplary method of determining whether a CR effect is reversible;

FIG. 9 illustrates an exemplary method of determining whether the effects of a CR mimetic are reversible;

Table 1 illustrates dietary compositions of the control diet program and the CR diet program.

Table 2 illustrates exemplary primary sequences for real time RT-PCR that can be used for some embodiments of the present invention;

Table 3 illustrates genes whose expression is affected by long-term CR;

Table 4 illustrates genes that display consistent changes in expression in response to CR administered for varying time points (e.g., a two-week CR, a four-week CR, an eight-week CR, and a long-term CR), such expression level changes being either consistently higher or lower than the control group, across all time points of CR; and

Table 5 illustrates genes whose expression is affected by short-term CR and long-term CR in different directions.

### DETAILED DESCRIPTION

In the following description, for purposes of explanation, numerous specific details are set forth in order to provide a thorough understanding of exemplary embodiments of the present invention. It will be evident, however, to one skilled in the art, that these embodiments may be practiced without these specific details. In other instances, specific structures and methods have not been described so as to not obscure the present invention. The following description and drawings are illustrative of the invention and are not to be construed as limiting the invention.

Throughout the discussion, the following terminologies are used. A control (CON) diet program or regimen refers to a normal feeding program having a normal number of calories (e.g., 93 kcal per week for a mouse test subject). A CR diet program refers to a dietary regimen with a reduced amount of calories (e.g., 77 kcal per week or 52 kcal per week for a mouse test subject). It is to be appreciated that the number of calories per week can be modified to adjust to what is considered normal for a particular test subject. A long-term caloric restriction (LT-CR) diet program refers to a reduced dietary regimen for a long duration of time, e.g., for more than eight weeks in the case of mice, or between about several months to about 36 months, or to about the end of life in some cases. A short-term caloric restriction (ST-CR) diet program refers to a reduced dietary regimen for a short duration of time, e.g., for about eight weeks or less than eight weeks in the case of mice. In certain situations, a diet program may be an ST-CR diet program which runs until about the end of life, when the ST-CR diet program is begun after a control diet program (e.g., a control diet program was administered to one or more animals in a test group for a long duration and the diet program for these animals was switched to a ST-CR diet program for the rest of the animals' lives). It is to be appreciated that the number of weeks or months that constitutes a short or long duration of time for a diet program or regimen can vary depending on experimental designs, test groups, mammalian species, etc.

A ST-CR group refers to a test group or a sample group that is subjected to a ST-CR diet program. A ST-CR group may further be divided into several sub ST-CR groups, for example a CR2 group, a CR4 group, and a CR8 group. A CR2 group refers to a ST-CR group that is subjected to the ST-CR diet program for a two-week duration. A CR4 group refers to a ST-CR group that is subjected to the ST-CR diet program for a four-week duration. A CR8 group refers to a ST-CR group that is subjected to the ST-CR diet program for an eight-week duration.

A short-term control (a ST-CON) group refers to a test group or a sample group that is subjected to a control diet program for a short duration of time relative to another diet program for a longer duration of time. Additionally, a CON8 group refers to a test group or a sample group that is subjected to a control diet program for a duration of 8 weeks.

A LT-CR group refers to a test group or a sample group that is subjected to a LT-CR diet program. A long-term control (LT-CON) group refers to a test group or a sample group that is subjected to a control diet program for a long duration of time.

A drug group refers to a test group or a sample group that is subjected to a dietary regimen for a duration of time (e.g., a predetermined period of time) that includes an administration of at least one intervention or a candidate intervention. An intervention can be a compound or a pharmaceutical agent (e.g., drug) that can be a potential CR mimetic. A candidate intervention can be a compound or a pharmaceutical agent (e.g., drug) that can be a potential CR mimetic or it may be a group of compounds or pharmaceutical agents. The drug group can also be divided into several sub-drug groups, for example, a 2 Wk-drug, a 4 Wk-drug, and an 8 Wk-drug, which represent different periods of exposure to an intervention (2 weeks, 4 weeks, and 8 weeks, respectively, in this example). A drug-withdrawn group refers to a test group or sample group that is subjected to withdrawal of the intervention that is administered to one of the groups as described above. The withdrawal of the intervention may be for a predetermined amount of time.

Exemplary embodiments are described with reference to specific configurations and techniques. The exemplary embodiments pertain to methods of analyzing effects induced by CR or a CR mimetic, and in some embodiments at different stages of CR treatment or CR mimetic treatment. The effects include at least one of the changes in gene expression levels (e.g., mRNA levels), changes in protein levels, changes in protein activity levels, changes in carbohydrate or lipid levels, changes in nucleic acid levels, changes in rate of protein or nucleic acid synthesis, changes in protein or nucleic acid stability, changes in protein or nucleic acid accumulation levels, changes in protein or nucleic acid degradation rate, and changes in protein or nucleic acid structures or function. The effects also include extending the longevity or life span of mammals (e.g., extending the longevity of mice). The following discussion focuses on several exemplary methods of identifying and categorizing genes that are expressed, not expressed, or otherwise altered (e.g., negatively or positively regulated) as induced by CR or a CR mimetic. The following discussion also focuses on extending the longevity of old mammals, for example, old mice, by subjecting the old mammals to a CR diet program in at least one stage.

A CR mimetic refers to a compound, a test compound, an agent, a pharmaceutical agent, or the like, that reproduces at least some effects induced by CR. It is to be appreciated by one skilled in the art that the exemplary methods are not limited to analyzing gene expressions that are affected by CR or CR mimetics but may include changes in physiological biomarkers such as changes in protein levels, protein activity, nucleic acid levels, carbohydrate levels, lipid levels, the rate of protein or nucleic acid synthesis, protein or nucleic acid stability, protein or nucleic acid accumulation levels, protein or nucleic acid degradation rate, protein or nucleic acid structures or functions, and the like.

Currently, CR that is started early, either early in life, or middle age, represents the best-established paradigm of retardation of aging in mammals. See for example, Weindruch, et al., The Retardation of Aging and Disease by Dietary Restriction, (C. C. Thomas, Springfield, Ill., 1988). The effects of CR on age-related parameters are broad. CR increases maximum lifespan, reduces and delays the onset of age-related disease, reduces and delays spontaneous and induced carcinogenesis, suppresses autoimmunity associated with aging, and reduces the incidence of several age-induced diseases (Weindruch, supra 1988).

Even though CR brings many beneficial effects to animals and humans, it is not likely that many will avail themselves of a CR lifestyle. As is known, it is difficult for any animal or human to maintain a diet program. Additionally, many believe that CR only acts incrementally or progressively to bring benefits to mammals such as extending lifespan and reducing and delaying the onset of age-related diseases. Such belief has not encouraged the use of CR to treat old mammals. Thus, there is a need to identify the dynamics of CR to determine whether CR can act rapidly such that CR can be beneficial to old mammals and not just young or middle-age mammals. There is also a need to identify, evaluate, and/or develop an intervention that is capable of mimicking some of the effects of CR, especially the beneficial effects, without the reduction of dietary calorie intake as required by CR diet programs. There is also a need to identify an intervention that can be administered to a mammal and that can rapidly reproduce the beneficial effects of CR. Identifying such interventions will enable treatments for mammals at almost all stages of life.

In addition, CR or CR mimetics may affect some genes in similar ways. Understanding the dynamics of the changes in gene expression in response to CR or CR mimetics is important since it may allow for more understanding of the behavior, structure and function of genes in a particular group. Furthermore, understanding the behavior, structure, and function of genes, how they interact within a group, and how they respond to CR and CR mimetics will enable the discovery of ways to regulate genes as a group. Thus, there is a need to identify the dynamics of changes of gene expression in groups of genes and to identify relatedness of genes to one another based on singular CR or CR mimetic treatments. When the dynamics of the changes in gene expression for groups of genes are better understood, it becomes easier and more efficient to regulate genes as a group or groups using fewer compounds and mechanisms.

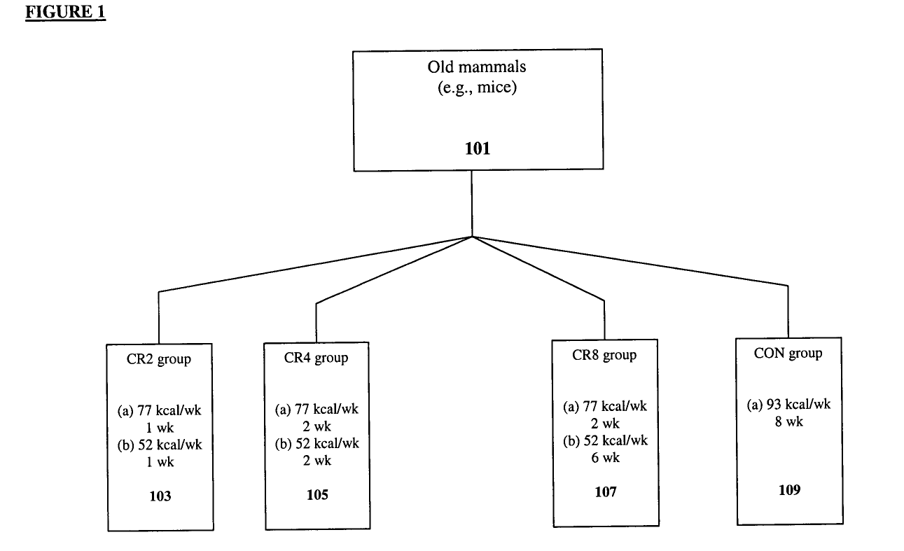

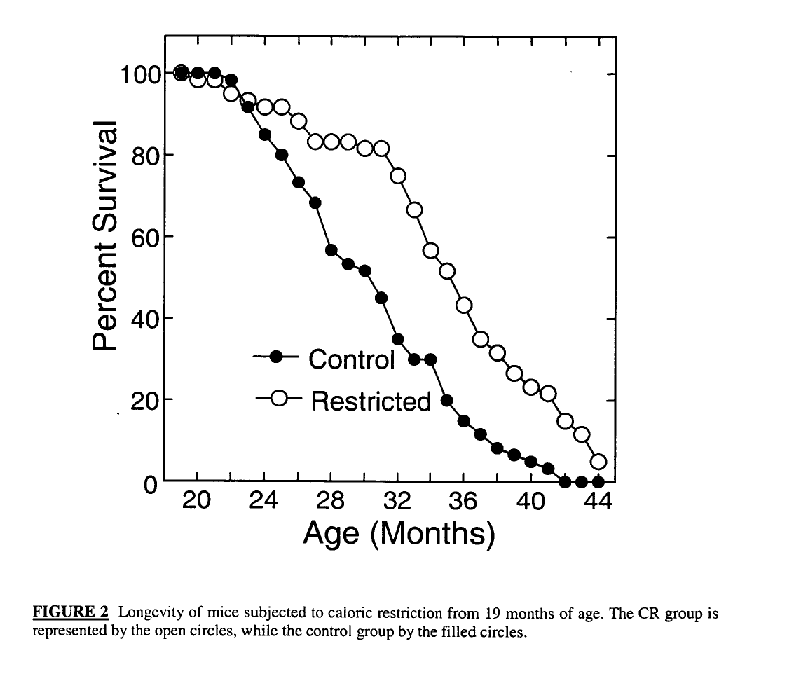

In one embodiment of the invention, the maximum lifespan of old mammals can be extended by treating the old mammals with a CR diet program. The old mammals can be gradually subjected to the CR diet program in stages, such as at least one stage. Treating the old mammals with the CR diet program in at least one stage should be done incrementally rather than suddenly (all at once). Thus, there is no sudden reduction in the number of calories in the mammals' diets. FIG. 1 illustrates an exemplary embodiment of treating old mammals 101, such as mice, with a CR diet program. As will be seen from below, treating old mammals with CR in stages can extend the maximum lifespan of the old mammals and bring other benefits of CR to the old mammals. In one embodiment, the old mammals 101 were divided into several groups, each of which underwent a CR diet program for a different amount of time. In one embodiment, the old mammals 101 were divided into a CR2 group 103, a CR4 group 105, a CR8 group 107, and a CON group 109.

In one embodiment, the old mammals 101 are male mice of the long-lived F1 hybrid strain B6C3F1. The old mammals 101 may be about 18 months old in the case of these mice. The mice were purchased from Harland (Indianapolis, Ind.). Each mouse from the CR2 group 103 was fed a 77 kcal per week CR diet for one week followed by a 52 kcal per week CR diet for another week. Each mouse in the CR4 group 105 was fed a 77 kcal per week CR diet for two weeks followed by 52 kcal per week CR diet for another two weeks. Each mouse in the CR8 group 107 was fed a 77 kcal per week CR diet for two weeks followed by a 52 kcal per week CR diet for six weeks. Each mouse in the CON group 109 was fed a 93 kcal per week control diet for eight weeks.

In one embodiment, the diet that was fed to each of the mice includes a semi-purified control diet in 1 gm pellets with a Control No. AIN-93M, Diet No. 505312, from BIO-SERV of Frenchtown, N.J., 08825. As illustrated in FIG. 1, for a particular group such as the CR2 group 103, CR4 group 105, and CR8 group 107, each mouse in these groups was subjected to a reduced diet program that ultimately resulted in a CR diet program that consisted of 52 kcal per week of a CR diet. It can be seen from FIG. 1 that the reduction was carried out in stages; for example, in the CR2 group 103, each mouse was subjected to the reduced diet by first going through the 77 kcal per week CR diet for one week and then finally to the 52 kcal per week CR diet for another week. The gradual reduction of calories in the diet, in stages, prevents the mice in each group from experiencing a sudden drop in caloric intake that may lead to death.

In addition, Table 1 illustrates the difference in dietary composition between the CR diet program and the control diet program. The control diet program consist of about 14 gm/100 gm diet casein, about 0.2 gm/100 gm diet L-cysteine, about 46.6 gm/100 gm diet cornstarch, about 15.5 gm/100 gm diet dextrinized cornstarch, about 10 gm/100 gm diet sucrose, about 4 gm/100 gm diet corn oil (Mazola), about 5 gm/100 gm diet cellulose, about 3.5 gm/100 gm diet mineral mix (AIN-76), about 0.3 gm/100 gm diet choline bitartrate, and about 1 gm/100 gm diet vitamin mix. The CR diet consist of about 23.3 gm/100 gm diet casein, about 0.3 gm/100 gm diet cysteine, about 29.5 gm/100 gm diet cornstarch, about 15.5 gm/100 gm diet dextrinized cornstarch, about 10 gm/100 gm diet sucrose, about 6.7 gm/100 gm diet corn oil, about 6.8 gm/100 gm diet cellulose, about 5.8 gm/100 gm diet mineral mix, about 0.4 gm/100 gm diet choline bitartrate, and about 1.7 gm/100 gm diet vitamin mix. Note that the 40% CR diet composition listed in Table 1 is for both the 52 kcal per week CR diet and the 77 kcal per week CR diet. The dietary composition for the diet in the reduction stage, where the diet includes a 77 kcal per week diet program, can be adjusted accordingly from the CR diet to obtain a 77 kcal per week diet. The CR diet was used for both 52 and 77 kcal per week CR diets.

In one embodiment, the effects of the CR diet program on the old mammals 101 are determined by comparing the results obtained from the CR diet program of the CR2 group 103, CR4 group 105, and CR8 group 107 to the results from the CON group 109. In one embodiment, the results include analyses of longevity of the mice in each of the groups CR2 group 103, CR4 group 105, and CR8 group 107, which were compared to the longevity of the mice in the CON group 109.

In some embodiments, parametric survival analyses were performed on the mice survival data. We assumed the data followed a Weibull distribution and the observed data were used to estimate the survival function. A change point regression analysis was also performed on the survival data to find the break points in the mortality data.

Unlike the conventional belief that CR acts progressively or incrementally, some embodiments of the present invention illustrate that CR rapidly affects the mammals that are subjected to CR, even at a later stage of their lives. For example, as illustrated in FIG. 1, a CR diet program was administered to the old mammals 101 (e.g., mice) for various lengths of time. In one embodiment, the rapid effects of CR in old mice and their similarity to the effects of LT-CR indicate to us that CR may have robust effects on life span even when initiated late in life. In one embodiment, a CR diet program (such as a long term CR diet program) was initiated in old mice (e.g., 19-month old mice) using the method shown in FIG. 1, just prior to the onset of accelerated mortality.

FIG. 2 illustrates that in one embodiment, the longevity of the old mice subjected to the CR diet program was compared to the longevity of the mice subjected to the control diet program. FIG. 2 illustrates that after the initiation of the CR diet program at 19 months of age, the mean time to death increased from 11.8±0.7 (SE) months in the control mice to 16.8±1.2 months (SE) in the CR mice (P=0.004), which is a 42% increase. These results indicate that CR initiated late in life is as effective at extending the remaining lifespan as CR initiated early in life. Late-life CR increased the mean and maximum lifespan by approximately 5 months,. In FIG. 2, the open circles represent the results obtained from a CR group (e.g. a LT CR group) and the filled circles represent the results obtained from the CON group 109. These groups can be summarized as the results of the CR diet administered to old mammals.

Conventional methods in the art have led to the conclusion that shifting mice at an advanced age to the CR diet program increased rather than decreased mortality. See for example, Forster, et. al., Genotype and age influence the effect of caloric intake on mortality in mice, FASEB J., (2003). The conventional methods shifted old mice that have been on a control diet program abruptly to the CR diet without progressively reducing the caloric intake over time (in stages). Rapid introduction of a CR diet program in old rodents results in elevated mortality. Furthermore, the average weight of the CR mice in these studies was too low, especially the DBA/2 mice, which did not show any effects of CR, suggesting that the CR diet programs used, imposed a state of overt starvation on these mice.

In one embodiment, a regression analysis revealed that the decrease in the mortality rate of the mice subjected to a CR diet program began within 2 to 3 months of initiating the CR diet program. A break point in the survival curve of the CR and control mice occurred at approximately 21.5 months of age as illustrated in FIG. 2. CR thus decreased the mortality rate by 3.1-fold between 21.5 and 31 months of age (p<0.001). Thereafter, the mortality rate of the CR mice approximated that of the control mice, but the lifespan was extended by about 5 months (p<0.001). These results indicate that CR very rapidly decelerated the underlying rate of aging, even though it was initiated late in life. Additionally, the CR and control mice died primarily of large tumors, mainly adenomas and carcinomas of the liver and lung (data not shown). Thus, late-life CR appeared to very rapidly delay and/or to decrease the onset and the progression of tumors.

Referring back to FIG. 1, in one embodiment, the results from the CR2 group 103, CR4 group 105, and the CR8 group 107 can be compared to LT-CR and control groups (e.g. LT-CON) to determine the shortest duration of time that the old mammals 101 need to be subjected to a CR diet program to obtain the benefits of CR. In some cases, a short duration such as a two-week duration as in the CR2 group 103 is sufficient to cause a positive effect on the gene expression profiles of the old mammals. In other cases, a longer duration is required, for example, a four-week duration (CR4 group 105) or an eight-week duration (CR8 group 107).

It should also be noted that for the embodiments discussed above and the those to be discussed below, control data can be obtained from a prior study, the results of which are recorded, as opposed to subjecting a control group of mice to a control diet program concurrently with the test groups of mice as illustrated in FIG. 1. Thus, the control data may be obtained from a previous administration of a control diet program to a control group of mice. This control data may be obtained once and stored for recall in later screening studies for comparison against the results in the later screening studies. Similarly, gene expression levels from CR mammals (or other types of measurements such as protein levels, nucleic acid levels, carbohydrate levels, lipid levels from CR) may be evaluated and recorded once and stored for recall in later screening studies for comparison against the results in the later screening studies. Of course, it is typically desirable for the prior stored studies to have a similar (if not identical) set of genes (or other parameters such as proteins) relative to the genes (or other parameters) in the later screening studies in order to perform a comparison against a similar set of genes or other parameters.

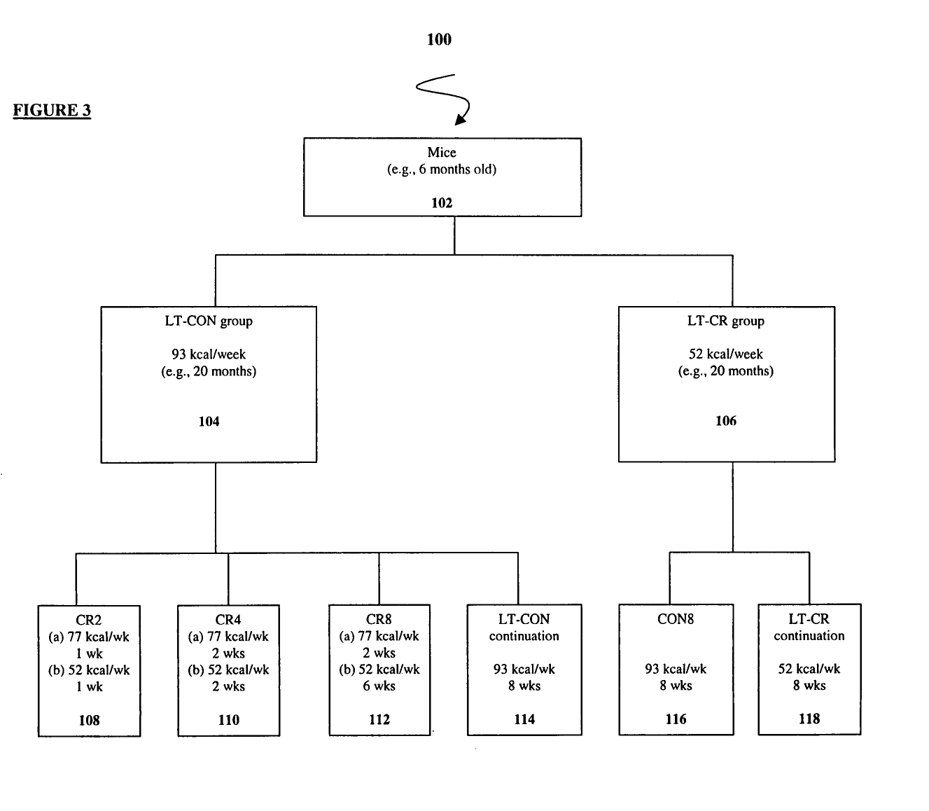

FIG. 3 illustrates an exemplary method 100 of subjecting a group of mammals to various dietary regimens. In one embodiment, the mammalian samples are mice. Male mice of the long-lived F1 hybrid strain B6C3F1 were purchased from Harland Laboratories, Indianapolis. For the first six months the mice were fed Rodent Diet No. 5001 (TMI Nutritional International LLC, Brentwood, Mo., 63044). At six months, all mice were individually housed. The 6-month old mice are indicated as mice group 102 as shown in FIG. 3. The mice in the group 102 were randomly assigned to two groups, an LT-CON group 104 and an LT-CR group 106. Each mouse in the LT-CON group 104 was subjected to a control diet program with a feeding of 93 kcal per week of a semi-purified control diet in 1 gm pellets for a long duration of time (e.g., 20 months in one group of mice). A complete list of diet ingredients or composition can be found in Table 1. Each mouse in the LT-CR group 106 was subjected to an CR diet program with a feeding of 52 kcal per week of the semi-purified diet for a long duration of time (e.g., 14 months in one case of mice). A complete list of the diet ingredients or composition can be found in Table 1.

In one embodiment, after 20 months of age, the mice from both the LT-CON group 104 and the LT-CR group 106 were subjected to a cross-over (or switching) experiment in which the mice in the LT-CR and the LT-CON groups were switched to opposite dietary regimens. The LT-CON group 104 was sub-divided into four groups, a CR2 group 108, a CR4 group 110, a CR8 group 112, and an LT-CON continuation group 114. Each mouse in the CR2 group 108 was subjected to a 77 kcal per week CR diet for 1 week followed by a 52 kcal per week CR diet for another 1 week. Each mouse in the CR4 group 110 was subjected to a 77 kcal per week CR diet for 2 weeks followed by a 52 kcal per week for another 2 weeks. Each mouse in the CR8 group 112 was subjected to a 77 kcal per week CR diet for 2 weeks followed by a 52 kcal per week CR diet for 6 weeks. Each mouse in the LT-CON continuation group 114 was maintained on the 93 kcal per week control diet for 8 weeks. Note that the LT-CON continuation group 114 simply refers to a group of mice that is subjected to the control diet for the additional amount of time such as 8 weeks. In one embodiment, the LT-CR group 106 was subdivided into two groups, a CON8 group 116 and an LT-CR continuation group 118. Each mouse in the CON8 group 116 was a LT-CR mouse subjected to a 93 kcal per week control diet for 8 weeks. Each mouse in the LT-CR continuation group 118 was maintained on the 52 kcal per week CR diet for 8 weeks. The LT-CR continuation group 118 simply refers to a group of mice that is subjected to a CR diet program for the additional amount of time such as 8 weeks.

In one embodiment, the results obtained for all of the test groups can be compared to each other (or to the control data previously recorded) to determine the effects of various CR diet programs and at various durations of time. In one embodiment, the effects discussed above may be determined by a biochemical measurement such as a gene expression level measurement. It is to be noted that measuring gene expression levels is only one method that can be used to determine the effects of CR or a candidate intervention. Other methods, such as those conventionally used for measuring specific protein activity levels, specific protein level changes, specific carbohydrate level changes, specific lipid level changes, and specific nucleic acid levels can be used. In one embodiment, specific mRNA levels obtained from the livers of mice from all of the various test groups were measured.

In one embodiment, total liver RNA was isolated from frozen tissue fragments by homogenization in TRI Reagent (Molecular Research Center, Inc., Cincinnati, Ohio, as described by the supplier) with an Ultra-Turrax (IKA Works, Inc. Wilmington, N.C.). mRNA levels were measured using Affymetrix M11K sets A and B high-density oligonucleotide arrays according to the standard Affymetrix protocol (Affymetrix, Santa Clara, Calif.). Briefly, cDNA was prepared from total RNA from each animal using Superscript Choice System with a primer containing oligo(dT) and the T7 RNA polymerase promoter sequence. Biotinylated cRNA was synthesized from purified cDNA using the Enzo BioArray High Yield RNA Transcript Labeling Kit (Enzo Biochem). cRNA was purified using RNeasy mini columns (Qiagen, Chatsworth, Calif.). An equal amount of cRNA from each animal was separately hybridized to MU11 sets A and B high-density oligonucleotide arrays. The arrays were hybridized for 16 h at 45° C. After hybridization, arrays were washed, stained with streptavidin-phycoerythrin, and scanned using a Hewlett-Packard GeneArray Scanner. Image analysis and data quantification were performed using the Affymetrix GeneChip analysis suite v5.0.

In embodiments where the Affymetrix Gene Chip analysis suite is used, each of the MU11K sets A and B comprises targets for more than 12,000 mouse Affymetrix unique identifiers. Each Affymetrix unique identifier is represented on the array by 20 perfectly matched (PM) oligonucleotides and 20 mismatched (MM) control probes that contain a single central-base mismatch. All arrays were scaled to a target intensity of 2500. The signal intensities of PM and MM were used to calculate a discrimination score, R, which is equal to (PM−MM)/(PM+MM). A detection algorithm that utilized R was used to generate a detection p-value and assign a Present, Marginal or Absent call using Wilcoxon's signed rank test. A detailed description of this method can be found in Affymetrix, I, New Statistical Algorithms for Monitoring Gene Expression on GeneChip Probe Arrays. Technical Notes 1, Part No. 701097 Rev.1 (2001), and Wilcoxon F., Individual Comparisons by Ranking Methods, Biometrics, 1:80-83 (1945). Only Affymetrix unique identifiers that were “present” in at least 75% of the arrays per experimental group were considered for further analysis. In addition, Affymetrix unique identifiers with signal intensities lower than the median array signal intensity in less than 75% of the arrays per experimental group were eliminated. These selection criteria reduced the raw data from 12,422 Affymetrix unique identifiers to only 2194 Affymetrix unique identifiers, which were considered for further analysis.

In one embodiment, to identify differentially expressed Affymetrix unique identifiers between any two groups, each of the samples (n) in one group was compared with each of the samples (p) in the other group, resulting in nxp pairwise comparisons. In one embodiment, n is equal to 3 or 4 and p is equal to 3 or 4. In one embodiment, the effects of LT-CR on gene expression were determined by comparing the results between the LT-CON continuation group and the LT-CR continuation group. In another embodiment, the effects of 2 weeks, 4 weeks, and 8 weeks of CR on gene expression were determined by comparing the results between the LT-CON continuation group 114 and the CR2 group 108, CR4 group 110, and CR8 group 112. The effects on gene expression produced by 8 weeks of control feeding were determined by comparing the results between the LT-CON group 114 and the CON8 group 116. In another embodiment, the effects of 2 weeks, 4 weeks, and 8 weeks of CR on gene expression were determined by comparing the results between the LT-CON continuation group 506 and the CR2 group 508, CR4 group 510, and CR8 group 512. The effects on gene expression of 2 weeks, 4 weeks, and 8 weeks of treatment with a candidate intervention will be determined by comparing the results between the LT-CON continuation group 506 and the 2 Wk-drug group 514, 4 Wk-drug group 516, and 8 Wk-drug group 518. In yet another embodiment, the effects of 2 weeks, 4 weeks, and 8 weeks of CR on gene expression in old mammals were determined by comparing the results between the CON group 109 and the CR2 group 103, CR4 group 105, and CR8 group 107.

The data were analyzed statistically using a method based on Wilcoxon's signed rank test. Difference values (PM−MM) between any two groups of arrays were used to generate a one-sided p-value for each set of probes. Default boundaries between significant and not significant p-values were used. See Affymetrix, I. New Statistical Algorithms for Monitoring Gene Expression on GeneChip Probe Arrays, mentioned above, for more details. The Affymetrix unique identifiers (known genes or ESTs) are considered to have changed expression if the number of increase or decrease calls was at least 75% of the pairwise comparisons. An average fold change, derived from all possible pairwise comparisons, of 1.5-fold or greater was considered significant. Empirically, these criteria for identifying gene expression changes can be reliably verified by methods such as Western blot, Northern blot, dot blot, primary extension, activity assays, real time PCR, and real time reverse transcriptase PCR (RT-PCR).

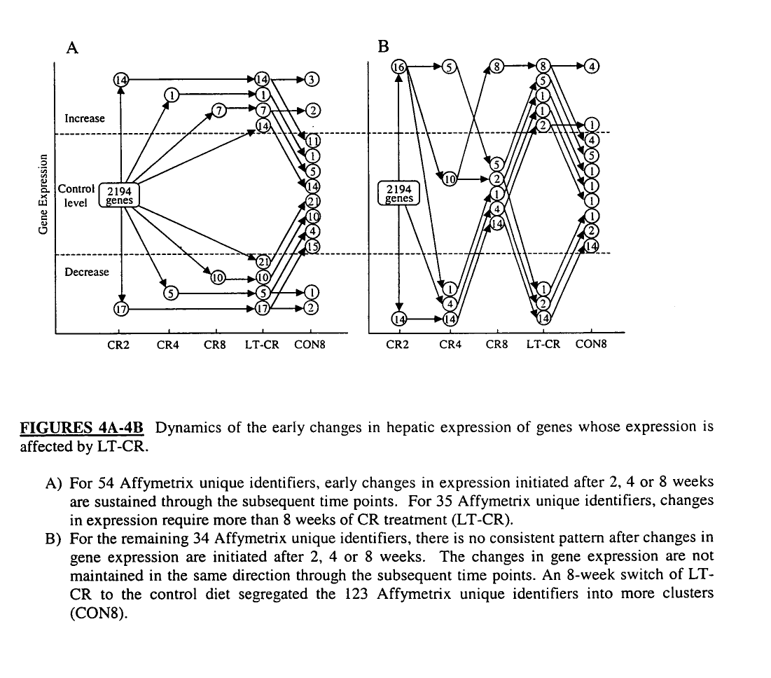

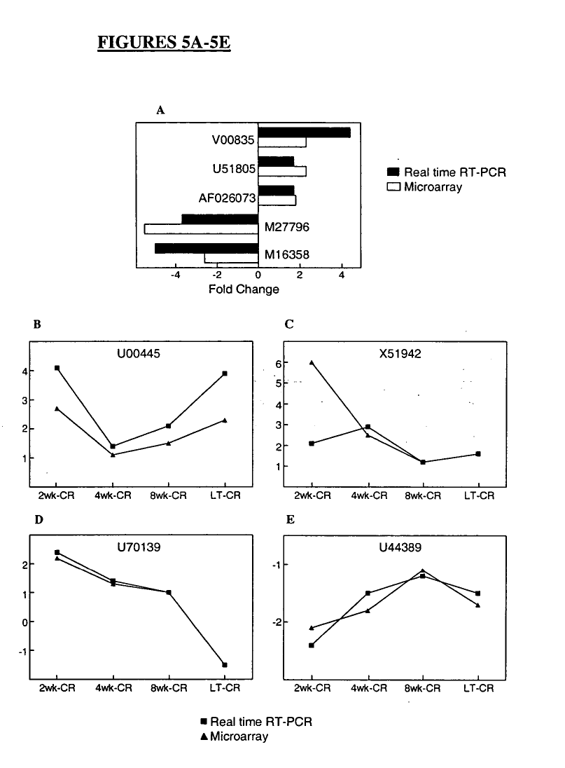

The results of the data are illustrated in Tables 3-5 and FIGS. 4A-4B. Gene names were obtained from the Jackson Laboratory Mouse Genome Informatics database as of Aug. 1, 2002. Gene names were obtained from the LocusLink and Affymetrix databases as of Jan. 23, 2003.

Tables 3-5 list some of the gene expression effects caused by the LT-CR diet program, CR diet program for 2 weeks, CR diet program for 4 weeks, CR diet program for 8 weeks, and the control diet program administered to mice that have been subjected to the LT-CR diet program and switched to the control diet program (e.g., CON 8 group 116) according to some embodiments. These gene expression effects are illustrated in terms of fold changes. In each of these tables, the Category/Gene column represents the category of the genes and the names of the genes and the Genebank column represents the Genebank identification number of the corresponding genes. In one embodiment, the numbers in the LT-CR column represent the average fold change in specific mRNA derived from all possible pairwise comparison (e.g., 16 possible pairwise comparisons) among individual mice from the LT-CR continuation group and the LT-CON continuation group 114 (e.g., number (n) of mice in each of these two groups is 4). The numbers in the CR2 column represent the average fold change in specific mRNA derived from all possible pairwise comparison (e.g., 16 possible pairwise comparisons) among individual mice from the CR2 group 108 and the LT-CON continuation group 114 (e.g., n=4). The numbers in the CR4 column represent the average fold change in specific mRNA derived from all possible pairwise comparison (e.g., 16 possible pairwise comparisons) among individual mice from the CR4 group 110 and the LT-CON continuation group 114 (e.g., n=4). The numbers in the CR8 column represent the average fold change in specific mRNA derived from all possible pairwise comparison (e.g., 16 possible pairwise comparisons) among individual mice from the CR8 group 112 and the LT-CON continuation group 114 (e.g., n=4). The numbers in the CON8 column represent the average fold change in specific mRNA derived from all possible pairwise comparison (e.g., 16 possible pairwise comparisons) among individual mice from the CON8 group 116 and the LT-CON continuation group 114 (e.g., n=4). Where there is no change in gene expression, an “NC” is denoted.

In one embodiment, the fold changes for each of the genes listed in Tables 3-5 are expressed in ratios. For each ratio, the numerator is the level of expression of each gene from the particular LT-CR, CR2, CR4, CR8, or CON8 group, and the denominator is the level of expression of that gene in the LT-CON continuation group. For example, the fold changes in gene expression caused by an LT-CR diet program is the ratio of the level of expression of each gene in the LT-CR continuation group divided by the level of expression of that gene in the LT-CON continuation group. The fold change in gene expression caused by a CR diet program for 2 weeks is the ratio of the level of expression of each gene in the CR2 group divided by the level of expression of that gene in the LT-CON continuation group. The fold change in gene expression caused by a CR diet program for 4 weeks is the ratio of the level of expression of each gene in the CR4 group divided by the level of expression of that gene in the LT-CON continuation group. The fold changes in gene expression caused by a CR diet program for 8 weeks is the ratio of the level of expression of each gene in the CR8 group divided by the level of expression of that gene in the LT-CON continuation group. The fold changes in gene expression caused by an 8-week switch to a control diet program after a LT-CR diet program is the ratio of the level of expression of each gene in the CON8 group divided by the level of expression of that gene in the LT-CON continuation group.

Table 3 lists genes which required more than 8 weeks of a CR diet program to change expression. Table 4 lists genes which responded early to a CR diet program and sustained their initial CR-induced expression levels at all subsequent time points for example, across 2 weeks, 4 weeks, 8 weeks, and longer than 8 weeks of a CR diet program; the genes in Table 4 may be referred to as “stables.” Table 5 lists genes which responded early to a CR diet program then returned to control levels briefly, before assuming their LT-CR expression level; the genes in Table 5 may be referred to as “oscillators.”

Validation of the relative gene expression levels was performed using real time RT-PCR. In one embodiment, the expression of a total of 9 genes randomly chosen from among the genes that have changed expression was examined by real time RT-PCR using total liver RNA purified from the mice used in the microarray studies. Total RNA was treated with DNase I (Ambion Inc., Austin, Tex.) and used to synthesize cDNA in a 20 μl total volume reaction. Briefly, 2 μg of total RNA were incubated with 250 ng random primer (Promega, Madison, Wis.) for 5 min at 75° C., and then on ice for 5 min. 2 μl of 0.1 M DTT, 4 μl of 5× buffer, 4 μl of 2.5 mM dNTP, 100 U (units) reverse transcriptase (Invitrogen, Carlsbad, Calif.), and 16.5 U RNase inhibitor (Promega) were added and incubated for 2 hr at 37° C. The reaction was stopped by boiling at 100° C. for 2 min. An identical reaction without the reverse transcriptase was performed to verify the absence of genomic DNA. All samples were reverse-transcribed at the same time and the resulting cDNA was diluted 1:4 in water and stored at −80° C.

Relative quantification with real-time, two-step real time RT-PCR was performed with a Quantitect SYBR Green PCR kit (Qiagen, Hilden, Germany) and using an ABI PRISM 7700 Sequence Detection System (Applied Biosystems, Foster City, Calif.), according to the manufacturer's instructions. Primers were designed using Netaffx analysis center and verified against the public databases to confirm unique amplification products (http://www.affymetrix.com/analysis/index.affx and http://www.ncbi.nlm.nih.gov), (see Table 2). Primers were chosen for transcription elongation factor A (S-II) 1 to amplify S-II in parallel with the gene of interest. S-II mRNA level is unaffected by a CR diet program. For each of the 9 genes in Table 2, real time RT-PCR was performed with each individual mRNA sample obtained from each mouse from each of the sample groups, for example, the LT-CON continuation group 114 (n=4), the LT-CR continuation group 118 (n=4), the CON8 group 116 (n=4), the CR2 group 108 (n=4), the CR4 group 110 (n=4), and the CR8 group 112 (n=4). Briefly, real time RT-PCR was carried out in a 25 μl volume containing 2 μl of diluted cDNA, 1×SYBR Green PCR Master Mix, 0.5 mM of each forward and reverse primers, and 0.5 unit uracil N-glycosylase. The reactions were incubated for 2 min at 50° C. to allow degradation of contaminating cDNA by uracil N-glycosylase, and 15 min at 95° C. to activate HotStarTaq DNA polymerase. Target amplification reactions were cycled 40 times with denaturation at 94° C. for 15 sec, annealing at 60° C. for 30 sec, and extension at 72° C. for 30 sec. To confirm amplification specificity, the PCR products from each primer pair were subjected to a melting curve analysis and subsequent agarose gel electrophoresis.

In one embodiment of the invention, the kinetics of the early effects by CR on gene expression are determined to gain insight into the mechanism of the rapid deceleration of aging and the reduction in the incidence of age-related pathology and diseases that result from shifting from a normal diet program to a CR diet program. In this embodiment, Affymetrix microarrays containing probes for approximately 12,000 Affymetrix unique identifiers were used to interrogate RNA samples purified from the old mice that were shifted from the life-long control feeding (e.g., the LT-CON group 104) to a CR diet program for 2, 4, and 8 weeks (e.g., the CR2 group 108, the CR4 group 110, and the CR8 group 112, respectively). In one embodiment, the gene expression profiles of the mice from these CR groups were compared to the gene expression profiles of the mice from the LT-CON group. In addition, the gene expression profiles of the mice from these CR groups were also compared to the gene expression profiles of the mice subjected to a LT-CR diet program (e.g., the LT-CR continuation group 118). Additionally, the gene expression profiles of mice that are shifted from a LT-CR diet program to a control diet program for a short duration of time (e.g., 8 weeks) are also determined by comparing the gene expression profiles of the mice from the CON8 group 116 to the mice from the LT-CON continuation group 114.

In one embodiment, of the approximately 12,000 Affymetrix unique identifiers interrogated, reliable signals for 2194 identifiers were obtained after data reduction. FIGS. 4A-4B and Tables 3-5 indicate that LT-CR diet programs altered the expression of 123 the Affymetrix unique identifiers (1% of the interrogated Affymetrix unique identifiers, 6% of the reporting Affymetrix unique identifiers). FIGS. 4A-4B and Tables 3-5 further indicate the effects of 2 to 8 weeks of CR diet program on the genes whose expression levels are monitored by these Affymetrix unique identifiers.

In FIGS. 4A-4B, the various durations of CR and control diets are represented on the x-axis with the indicators CR2, CR4, CR8, LT-CR, and CON8. CR2, CR4, and CR8 indicate the gene expression results for the mice that were subjected to a CR diet program for 2 weeks, 4 weeks, and 8 weeks, respectively (e.g., the CR2 group 108, the CR4 group 110, the CR8 group 112, respectively, of FIG. 3). LT-CR indicates the gene expression results for the mice that were subjected to a CR diet program for a long duration of time, e.g., 22 months (e.g., the LT-CR continuation group 118, FIG. 3). CON8 indicates the gene expression results for the mice that were subjected to a shift to the control diet program (e.g., for 8 weeks) after being subjected to a CR for a predetermined duration of time (e.g., 20 months) (e.g., the CON8 group 116, FIG. 3). The results in FIGS. 4A-4B demonstrate that following the onset of the CR diet program, there is a rapid and progressive shift toward the gene expression profile associated with the LT-CR diet program.

In one embodiment, the responding genes are divided into three temporal classes termed early responders, middle responders, and late responders. The early responders are those genes that changed expression between 2 to 4 weeks of the CR diet program. The middle responders are those genes that changed expression between 4 and 8 weeks of the CR diet program. The late responders are those genes that required more than 8 weeks of the CR diet program to respond (e.g., the genes that changed expression in the LT-CR diet program but did not change expression in the CR2, CR4, and CR8 groups). Among the early and middle responders, some genes sustained their CR-induced expression levels at all subsequent time points. For example, as illustrated in FIG. 4A, clusters of genes remained in the increased or decreased levels from CR2, to CR4, to CR8, and to LT-CR. These genes may be referred to as stables. Among the early responders, some genes returned to control levels briefly, before assuming their LT-CR expression levels (FIG. 4B). These genes may be referred to as oscillators.

As illustrated in FIGS. 4A-4B, 71 of the 123 Affymetrix unique identifiers (58%) were early responders, and these were nearly evenly divided between stables and oscillators (37 stables and 34 oscillators). 77 of the Affymetrix unique identifiers (14%) were middle responders (all stables), and 35 Affymetrix unique identifiers (28%) were late responders. These results indicate that the majority of the genes responded early to the effects of CR, and that the stables somewhat outnumber the oscillators.

Quantitative change in the activity of specific genes can control the rate of aging and/or age-related diseases. For example, quantitative change in the activity of specific genes can decelerate the rate of aging and/or age-related diseases. CR diet programs can alter the expression of genes that affect or decelerate the rate of aging or age-related diseases. Insight into the mechanism or the dynamics of the changes of the genes enables a more complete understanding of the relationship and effects of a CR diet program or a CR mimetic and the observed deceleration of aging and reduction in incidence of age-related pathology and diseases. At least some embodiments of the present invention indicate that the deceleration of aging and/or beneficial effects on age-related diseases caused by a CR diet program or a CR mimetic is rapid.

The early, middle, and late CR responsive genes are likely regulated by different signal transduction pathways. Combinatorial interactions among the components of the pathways may induce or repress genes at each time point. In one embodiment, the pathways involved are further analyzed using motif discovery.

Switching sample groups to different diet programs according to some of the embodiments discussed above, (e.g., FIG. 3) allows for motif discovery. For instance, the switching or crossover feeding distinguished some genes whose expression was altered by LT-CR but not by CR2, CR4, or CR8. The differences in the dynamics of changes in mRNA levels suggest that CR involves multiple complex molecular mechanisms in its effects on gene expression. Genes assemble into clusters most likely because of similarities in the molecular mechanisms of their regulation. For example, several genes may have a common regulatory factor (e.g., enhancer sequences) or a common signal transduction pathway, and these common features are revealed through the gene clusters identified as a result of switching the diet programs. Thus, the switching of diet programs allows for motif discovery and allows for genes to be categorized.

As is apparent from FIGS. 4A-4B, genes are fractionated into clusters as certain genes are similarly affected by a particular dietary regimen. Genes in the same cluster are likely to be transcriptionally co-regulated and their promoter regions can be analyzed for the presence of shared sequence motifs. Motif discovery begins by identifying genes that are co-regulated under different conditions by CR. Genes which respond in the same way to given physiological conditions are grouped together. For example, as illustrated in FIG. 4A, genes which are responsive to CR2 and LT-CR form 2 clusters (14, 17); genes which are responsive to CR4 and LT-CR form 2 clusters (1, 5); and genes which are responsive to CR8 and LT-CR form 2 clusters (7, 10). Also as illustrated in FIG. 4A, genes which are only responsive to LT-CR form 2 clusters (14, 21). Switching the mice to an 8-week control diet program following a LT-CR diet program further subdivides genes into 12 clusters (3, 2, 11, 1, 5, 14, 21, 10, 4, 15, 1, 2). The results from FIGS. 4A-4B indicate that the expression of different genes can be stimulated or inhibited by the same regulatory factors and signal transduction systems.

The most parsimonious explanation for the co-behavior of each of these clusters of genes is that they are co-regulated by the same signal transduction pathway. Gene regulation in eukaryotes mainly involves transcription factors binding to short DNA sequence motifs located upstream of the coding region of genes. Thus, the upstream sequences of a set of co-regulated genes can be analyzed for shared cis-regulatory motifs (short DNA sequences). These known or unknown DNA sequence motifs (regulatory motifs) common to gene clusters are putative binding sites for transcription factors. Algorithms such as AlignACE have been used to identify known and novel sequence motifs based on gene expression data from microarray experiments. Thus, promoter comparison within clusters and genes can identify potential binding sites for known or novel transcription factors that might control gene expression during CR. Knowledge of the identity of the transcription factors bound by the putative regulatory motifs will suggest which signal transduction systems may be responsible for the regulation of the genes by CR. The signal transduction systems responsible for gene regulation by many transcription factors are known. The signal transduction systems responsible for regulation of the activity of other transcription factors, including novel transcription factors which may be identified, may be determined experimentally.

In some embodiments, drugs that alter the activity of identified, known signal transduction systems may be possible candidate CR mimetics. In other embodiments, potential CR mimetics that alter the activity of the identified signal transduction systems may be identified experimentally by monitoring some feature of the activity of the signal transduction system. This feature might be, for example, the phosphorylation or other modification of the structure or activity of a protein or changes in the activity of a specific gene. In this way, motif discovery may aid in the discovery or development of pharmaceuticals capable of mimicking the life- and health-span extending effects of CR.

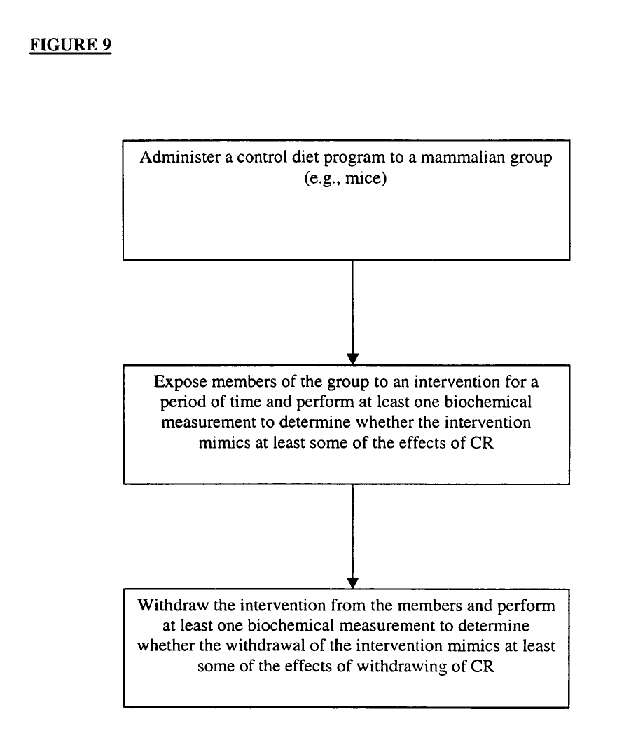

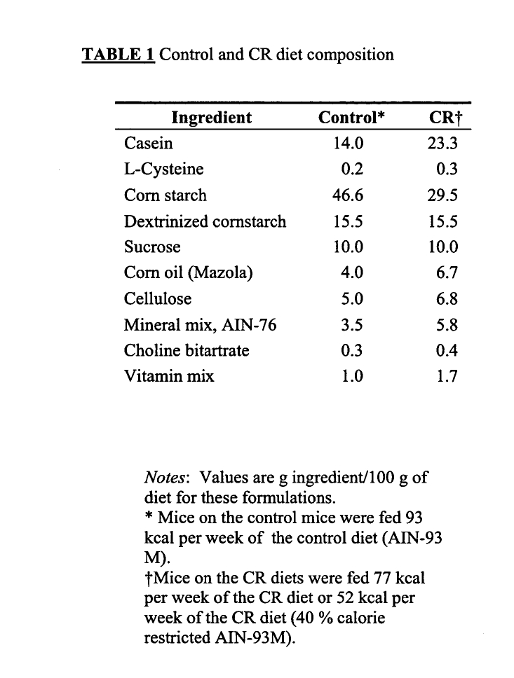

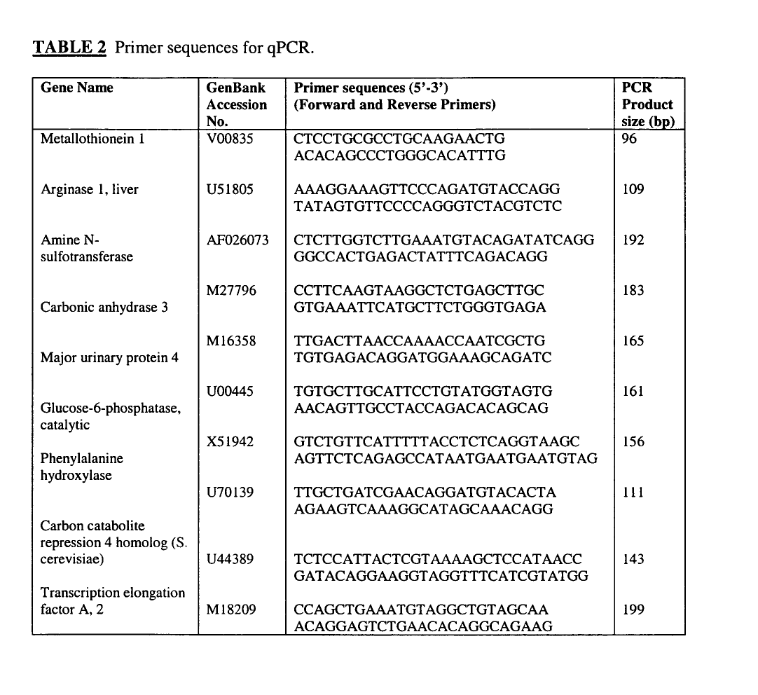

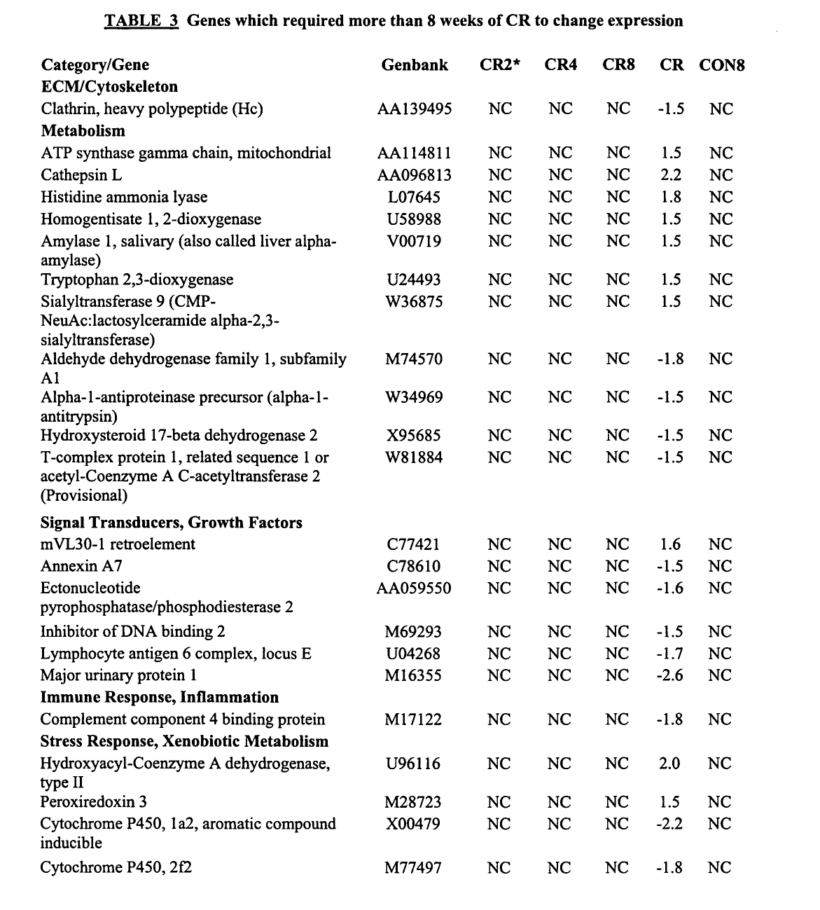

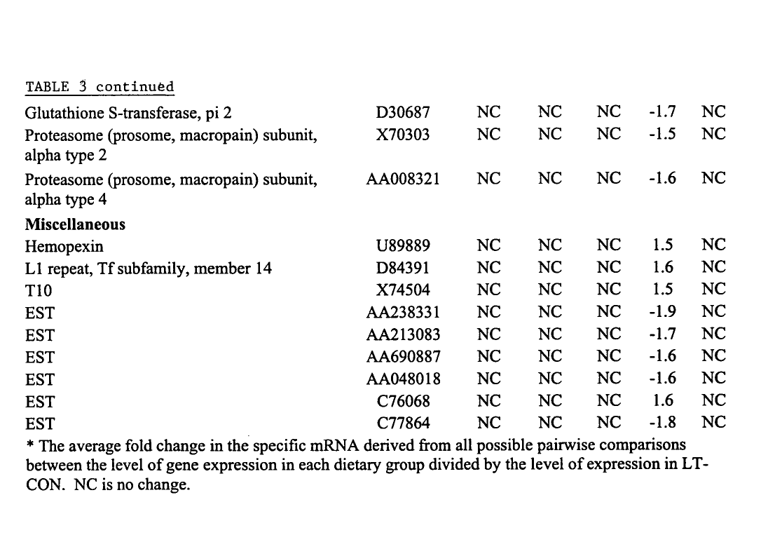

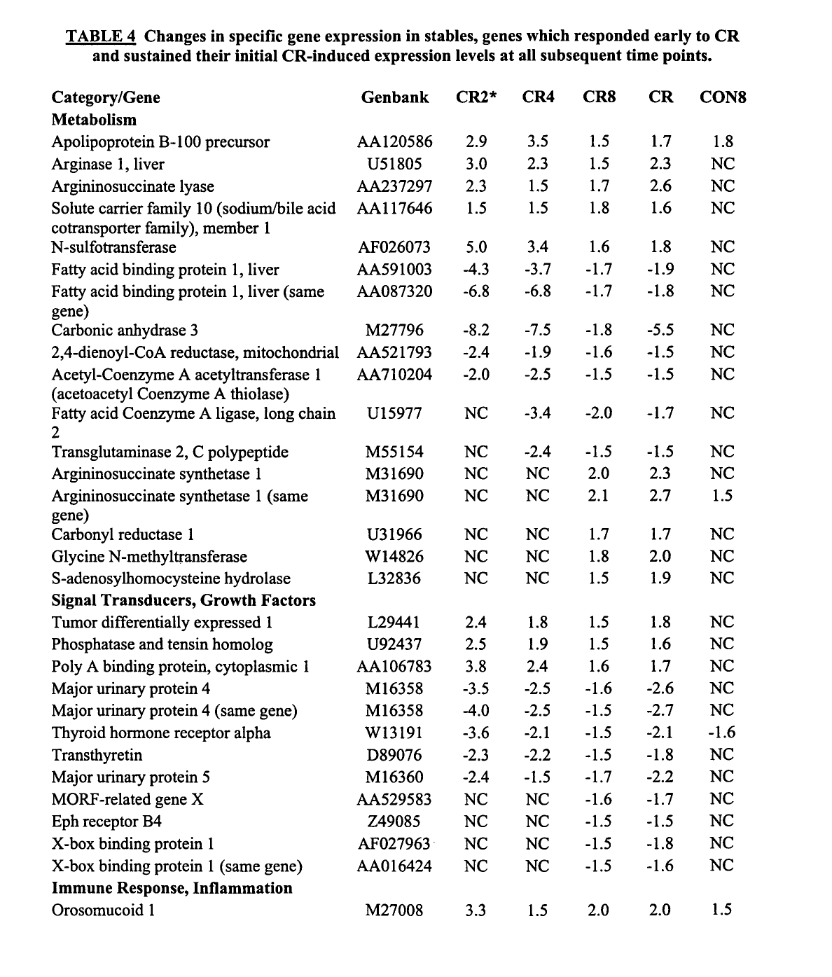

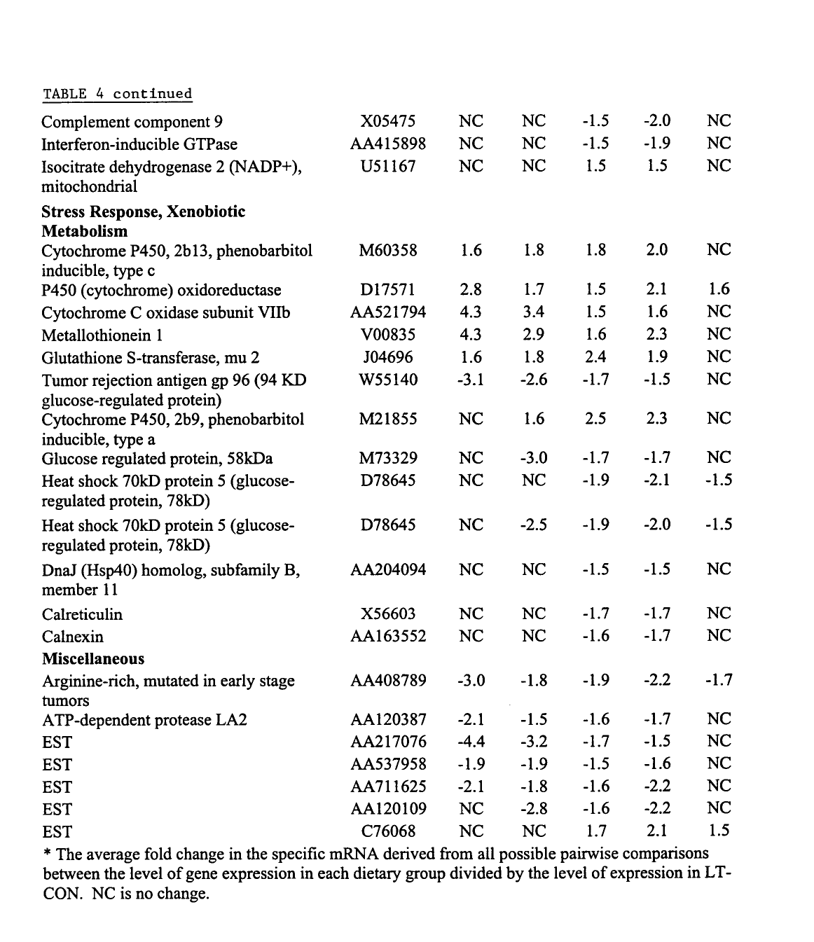

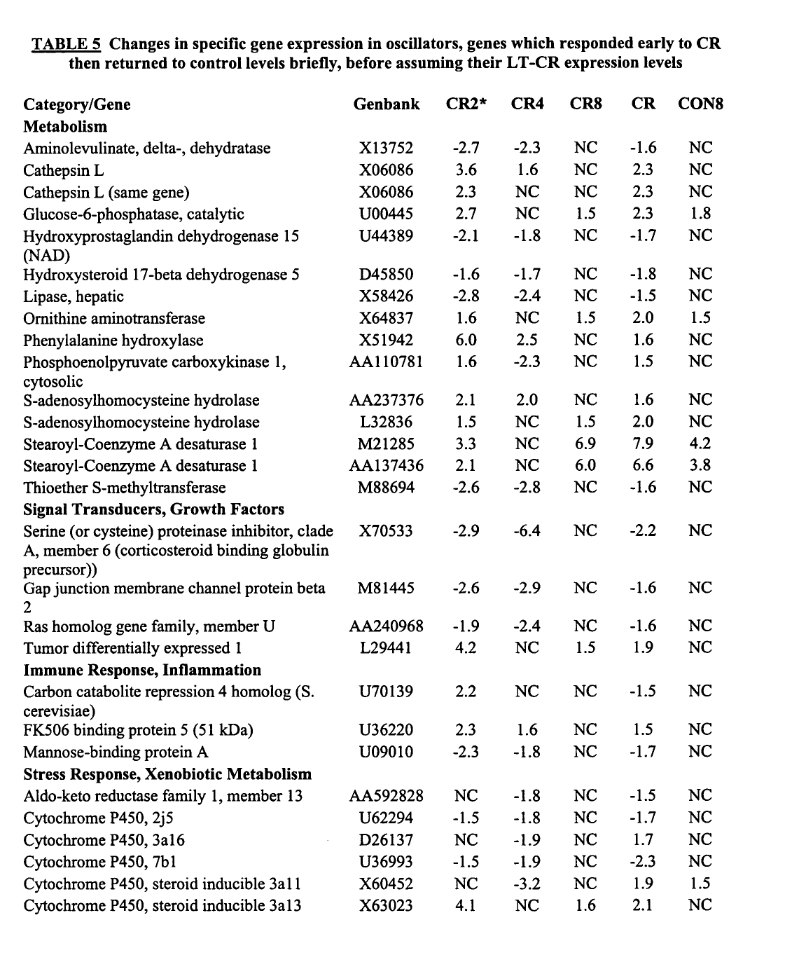

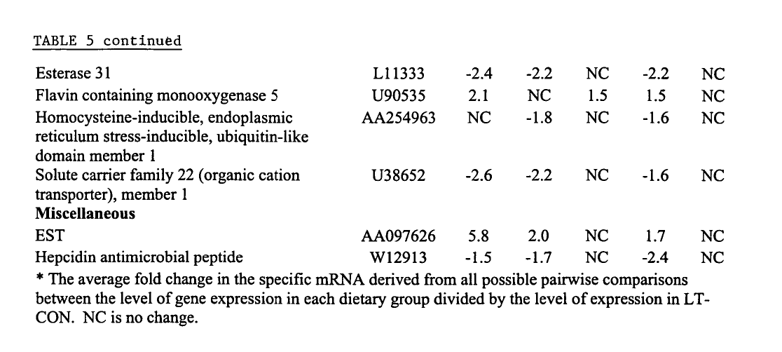

In one embodiment, the effects of the transition from a CR diet program to a control diet program are determined. Using some of the embodiments discussed above (for example, the embodiments discussed with reference to FIG. 3), it was determined that many, if not most, of the gene expression levels of the mice that were switched to a control diet after a period of being subjected to a CR diet program returned to the control expression levels (FIGS. 4A-4B). The control expression levels are the gene expression levels of the genes from mice that are subjected only to a control diet program. These embodiments thus provide methods to directly study the transition of the CR diet program to the control diet program. Switching the mice from the CR diet program (e.g., the LT-CR group 106) to the control diet program (e.g., the CON8 group 116) revealed that many genes that were affected by CR returned to the control expression levels after the switch. In one embodiment, 110 of the 123 (90%) Affymetrix unique identifiers that were affected by the LT-CR diet program returned to control expression levels. FIGS. 4A-4B indicate that all of the late responsive genes were shifted from their LT-CR expression levels to control expression levels (see for example, the cluster with 14 genes and the cluster with 21 genes at the LT-CR mark which were shifted to the control expression levels at the CON8 mark in FIG. 4A). The results in FIGS. 4A-4B indicate that many of the effects of CR are reversible. These results suggest that even though the late responder genes required more than 8 weeks of the CR diet program to change expression, they were rapidly responsive to changes in caloric intake. These results further indicate that most genes respond rapidly to changes in caloric intake. These results also indicate that a method may be used, as shown in FIG. 9 and as discussed further herein, to test a candidate CR mimetic (e.g., a candidate intervention) to determine if the effects of the candidate CR mimetic are reversible.

In one embodiment, switching to the control diet program for 8 weeks after a CR diet program provides a method of fractionating genes that are responsive to CR into defined clusters amenable to further study.

In one embodiment, the genes that changed expression due to various CR diet programs at various time points were clustered into functional classes including (1) carbohydrate, fat, and protein metabolism; (2) growth factor and signal transduction; (3) cytoprotective stress-responses, oxidative and reductive xenobiotic metabolism, and chaperones; and (4) immune response and inflammation. Tables 3-5 include the gene expression results of the genes belonging to these classes and how the gene expression of these genes is affected by a CR diet program at 2, 4, and 8 weeks, by an LT-CR diet program, and by a switch to a control diet program after a CR diet program.

In one embodiment, the genes in the carbohydrate, fat, and protein metabolism class that are altered by a CR diet program are listed in Tables 3-5. Of the 26 metabolic genes discussed below, 23 were early or middle responders. Thus, the initial phases of the metabolic transition from the control to the CR state occur essentially completely during the first 8 weeks of the CR diet program. Some oscillators return to control expression levels before reaching their LT-CR expression levels. Consistent with their rapid shift in response to the CR diet program, 23 of the 26 genes reverted to control expression levels after only 8 weeks of control diet program.

As illustrated in Table 4, CR induced the expression of three urea cycle enzymes, arginase 1, argininosuccinate lyase, and argininosuccinate synthetase 1. Nitrogen derived from amino acid catabolism in the periphery is disposed of from the liver via the urea cycle. Thus, CR enhances the disposal of nitrogen in the liver. CR also increased the expression of cathepsin L (Table 3), phenylalanine hydroxylase (Table 5), homogentisate 1, 2-dioxygenase (Table 3), ornithine aminotransferase (Table 5) and histidine ammonia lyase (Table 3). These genes are involved in amino acid degradation to provide substrates for gluconeogenesis. Consistent with these effects, CR induced expression of phosphoenolpyruvate carboxykinase 1 (Table 5) and glucose-6-phosphatase (Table 5), which are the key gating enzymes of gluconeogenesis. These results indicate that CR enhances the enzymatic capacity of the degradation of amino acids for energy production. Because the weights of the animals are approximately at steady state, CR apparently enhances the turnover and resynthesis of whole body protein. It is to be noted that such effects are observed not only during fasting, but also in the hours following feeding.

Continuing with the metabolism class, CR positively affected the function of lipid metabolism. CR decreased the expression of acetyl-CoA acetyltransferase 1 (Table 4), fatty acid Coenzyme A ligase, long chain 2 (Table 4), 2,4-dienoyl-CoA reductase mitochondrial (Table 4), liver fatty acid binding protein 1 (Table 4), and hepatic lipase (Table 5). The decrease in the expression of these genes should reduce the enzymatic capacity for lipid biosynthesis and metabolism. The decrease in the expression of these genes may account for the decrease in serum triglycerides observed in rodents that were subjected to a CR diet program.

Still continuing with the carbohydrate, fat, and protein metabolism class, CR also increased the expression of apolipoprotein B-100 (Table 4), which is a major component of low density lipoprotein and very low density lipoproteins. The increased expression of this gene also enhances its role in the distribution of hepatic lipid to other tissues for use as fuel. Additionally, CR also decreased the expression of hydroxysteroid 17-beta dehydrogenase 5 (Table 5) and hydroxysteroid 17-beta dehydrogenase 2 (Table 3), which are enzymes responsible for the biological inactivation of testosterone. The decreased expression of these genes may help in maintaining or controlling the level of testosterone in aging mammals. For instance, the decreased expression of these genes may account for the higher testosterone levels seen in old rodents that were subjected to a CR diet program compared to old rodents that were not subjected to a CR diet program.

Still continuing with the carbohydrate, fat, and protein metabolism class, CR also decreased the expression of the mRNA for hydroxyprostaglandin dehydrogenase 15 (Table 5). This gene catalyzes the initial step in the inactivation of circulating prostaglandins, including prostaglandin E(2). The inactivation of the circulating prostaglandins may be a compensatory response to a reduced and/or age-associated systemic inflammation in animals that were subjected to a CR diet program.

Continuing with the carbohydrate, fat, and protein metabolism class, CR beneficially affected methylation activity. CR decreased the expression of thioether S-methyltransferase (Table 5), which catalyzes the transfer of the methyl group from S-adenosylmethionine to sulfur, selenium, or tellurium compounds. CR increased the expression of S-adenosylhomocysteine hydrolase (Table 5), which hydrolyzes the S-adenosylhomocysteine (SAH) formed after donation of the methyl group of S-adenosylmethionine (SAM) to a methyl acceptor. CR also increased the expression of glycine N-methyltransferase (Table 5), which catalyzes the methylation of glycine by S-adenosylmethionine to form N-methylglycine (sarcosine) and SAH. Glycine N-methyltransferase and S-adenosylhomocysteine hydrolased together can control the SAM to SAH ratio. Increased SAH leads to decreased transmethylation of phospholipids, proteins, small molecules, DNA and RNA. Decreased methylation is generally associated with enhancement of transcriptional activity and differentiation.

In one embodiment, the genes in the class of signal transducers and growth factors that were affected by a CR diet program are listed in Tables 3-5. As illustrated, CR altered the expression of genes associated with cell growth and proliferation. In one embodiment, CR decreased the expression of lymphocyte antigen 6 complex, locus E (Table 3), Ras homolog gene family, member U (Table 5), and inhibitor of DNA binding 2 gene (Table 3).

CR also decreased the expression of two genes associated with angiogenesis, Eph receptor B4 (Table 4) and ectonucleotide pyrophosphatase/phosphodiesterase 2 (Table 3). Moreover, CR induced the expression of phosphatase and tensin homolog gene (Table 4), which has a tumor suppression activity. Thus, CR appears to enhance anti-proliferative growth control.

CR also decreased the expression of transthyretin (Table 4), and thyroid hormone receptor alpha (Table 4), which are the major thyroid hormone carrier proteins in rodents. The decreased expression of these genes leads to reduced thyroid hormone signals in animals and humans that are subjected to a CR diet program. The reduction of thyroid hormone signal in turn reduces a diverse set of energy utilization-related processes, including the metabolism of lipids, carbohydrates, and proteins, and oxygen consumption.

The results above indicate that CR extends the longevity and delays the onset of age-related diseases in mammals. Furthermore, these results indicate that CR is also effective in treating old mammals (as well as younger mammals) and that CR acts rapidly to bring the benefits of CR to the mammals.

In one embodiment, the results in Tables 3-5 also indicate that CR altered the expression of chaperone proteins. Most proteins require interactions with molecular chaperones for their biosynthesis, maturation, processing, transport, secretion, and degradation. It has been found that the mRNA and protein levels of most endoplasmic-reticulum chaperones increase with age. CR decreases the caloric intake in the liver and other tissues thus decreasing the mRNA and protein levels of most endoplasmic reticulum chaperones. The linkage between caloric intake and chaperone expression may match protein folding, assembly, and processing capacity to the level of insulin stimulated protein biosynthetic activity. Elevated chaperone expression also decreases apoptotic responsiveness to genotoxic stress. Chaperones repress apoptosis through both the endoplasmic stress and the mitochondrial apoptosis signaling pathways. The anti-cancer benefits of CR may result from the fact that CR reduces endoplasmic reticulum chaperone levels and enhances apoptosis in liver and other cell types. In contrast, in non-dividing cells, such as neurons, CR appears to induce chaperone expression, thereby enhancing cell survival.

In one embodiment, the results in Tables 3-5 also indicate that CR altered the expression of genes in the xenobiotic metabolism class. CR differentially regulated the expression of a number of phase I and II enzyme genes. For example, CR enhanced the expression of N-sulfotransferase (Table 4), flavin-containing monooxygenase 5 (Table 5), several cytochrome P450 isozymes and glutathione S-transferase, mu2 (Table 4). Examples of some of the cytochrome P450 isozymes that are enhanced by CR include cytochrome P450, 3a16 (Table 5), cytochrome P450, steroid inducible 3a11 (Table 5), cytochrome P450, steroid inducible 3a13 (Table 5), cytochrome P450, 2b13, phenobarbitol inducible, type c (Table 4), cytochrome P450, 2b13, phenobarbitol inducible, type a (Table 4), and cytochrome P450 oxidoreductase (Table 4). The increased expression of these genes may enhance drug metabolization and detoxification functions of the liver. It is to be noted that many of these enzymes also can enhance toxicity and carcinogenicity of some substrates. Thus, the effects that CR will have on xenobiotic metabolism are dependent on the xenobiotic environment. The physiological impact of the CR on the decreased expression of cytochrome p450, 1a2 (Table 3), cytochrome p450, 2f2 (Table 3), cytochrome p450, 2j5 (Table 5) and cytochrome p450, 7b1 (Table 5), and glutathione S-transferase, pi 2 (Table 3) is difficult to predict. For example, CR is reported to induce Cyp2e1, which leads to 2.5-fold greater bioactivation thioacetamide, a potent hepatotoxin and carcinogen. However, CR also increased resistance to thioacetamide hepatotoxicity, perhaps by enhancing the rate of liver apoptosis and regeneration. Thus, through differential gene regulation CR may strike a balance between toxin and carcinogen activation and deactivation, and cellular growth and apoptosis.

In one embodiment, to verify or validate that the genes responded as indicated in FIGS. 4A-4B the expression of 9 randomly chosen genes was monitored by a quantitative PCR (e.g., real time RT-PCR) as illustrated in FIGS. 5A-5B. In every case, quantitative PCR confirmed the changes found by microarray gene expression profiling at each of the CR time-points.

FIGS. 5A-5E illustrate the results of validating the 9 randomly chosen genes (with gene names V00835, U51805, AF026073, M27796, M16358, U00445, X51942, U70139, and U44389) using the real time RT-PCR. As illustrated in FIGS. 5A-5E, real time RT-PCR confirmed the changes found by microarray gene expression profiling for each of the 9 chosen genes. As can be seen from this figure, the fold changes are in the same direction and are substantially similar in the amount of the fold changes.

FIG. 5A illustrates validation of some of the genes that change with LT-CR (see genes V00835, U51805, AF026073, M27796, and M16358). The open bars represent the microarray data and the solid bars represent the real time RT-PCR data. The real time RT-PCR data represent the fold changes in the specific mRNA derived from comparing the results between the mice from the LT-CR continuation group and mice from the LT-CON continuation group measured using real time RT-PCR. The microarray data represent the average fold changes in the specific mRNA derived from all possible pairwise comparisons among individual mice from the LT-CR continuation group and the LT-CON continuation group.

FIGS. 5B-5E illustrate some of the genes that have changes in expression that fluctuates across the various time points. The triangles represent the microarray data and the squares represent the real time RT-PCR data. FIG. 5B compares the microarray data and the real time RT-PCR data for the gene U00445. FIG. 5C compares the microarray data and the real time RT-PCR data for the gene X51942. FIG. 5D compares the microarray data and the real time RT-PCR data for the gene U70139. FIG. 5E compares the microarray data and the real time RT-PCR data for the gene U44389.

The results in FIGS. 5A-5E indicate that the use of the microarray analytical methods that are used for some of the embodiments of the present invention reliably identified genes that change expression. The validation of the microarray analytical methods also insures that the complexity of the response of the genes did not arise simply from the stringency of the selection criterion. As can be seen from these figures, in no case did the assignment increase, or decrease in fold change in the gene expression levels arise from close calls in the selection criterion.

As shown in FIGS. 5A-5B, the fold changes in the 9 randomly chosen genes confirmed the data obtained using the microarray methods. Thus, the expression patterns shown in FIGS. 4A-4B represent the true kinetics of the response to CR in old mice.

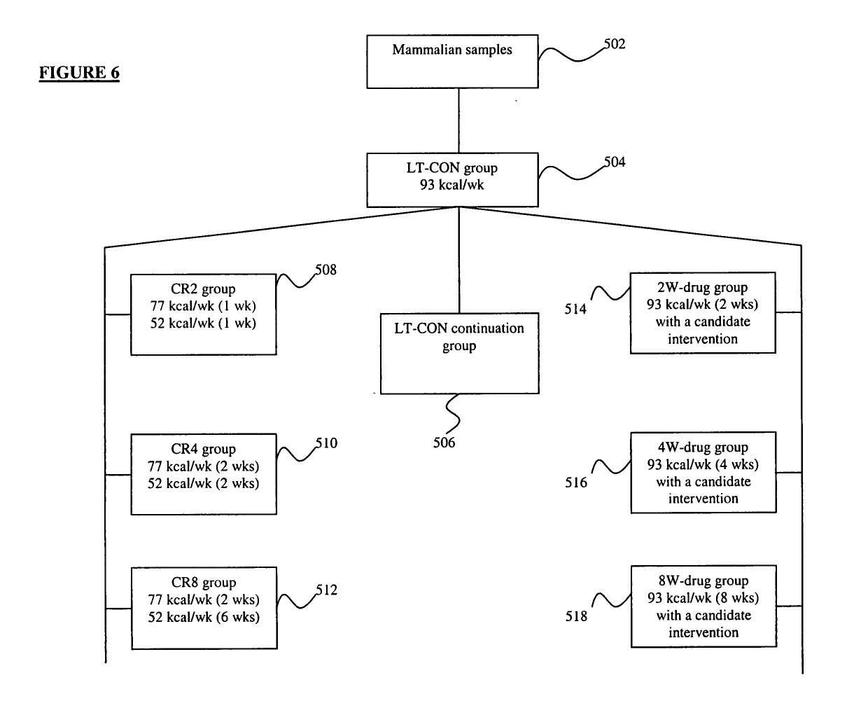

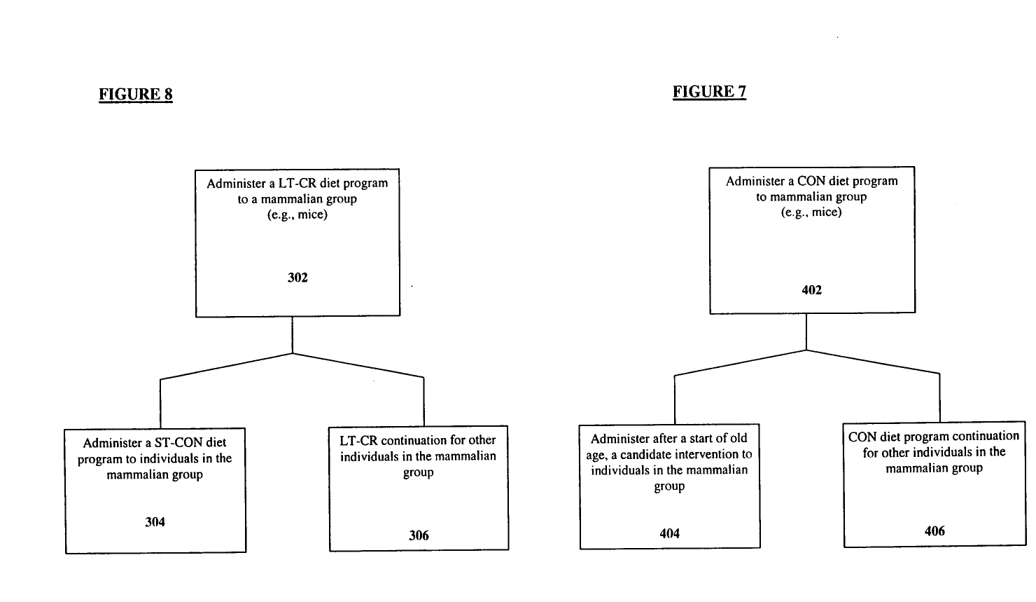

Using some of the techniques previously described, a candidate intervention can be discovered and analyzed. FIG. 6 illustrates that in one embodiment, a candidate intervention that is a CR mimetic candidate or a potential CR mimetic can be administered to a group of mammals for different lengths of time. This figure illustrates that in one embodiment, mammalian samples 502 (e.g., mice) are subjected to an LT-CON diet program generating an LT-CON group 504. Each member of the mammalian samples 502 is fed a 93-kcal per week control diet for a predetermined duration of time, e.g., 20 months, to generate the LT-CON group 504. In one embodiment, the 93-kcal per week control diet is a normal diet program in the embodiments where the mammalian samples are mice. The normal number of calories may change accordingly depending on the type of the mammalians samples.

After the predetermined duration of time, the members in the LT-CON group 504 are divided into several groups, which include an LT-CON continuation group 506, a CR2 group 508, a CR4 group 510, a CR8 group 512, a 2 Wk drug group 514, a 4 Wk drug group 516, and an 8 Wk drug group 518. Each of the members in the CR2 group 508 is subjected to a CR diet program that reduces the number of calories from 93 kcal per week to 77 kcal per week for 1 week followed by a 52 kcal per week for another 1 week. Each of the members in the CR4 group 510 is subjected to a CR diet program that reduces the number of calories from 93 kcal per week to 77 kcal per week for 2 weeks followed by a 52 kcal per week for another 2 weeks. Each of the members in the CR8 group 508 is subjected to a CR diet program that reduces the number of calories from 93 kcal per week to 77 kcal per week for 2 weeks followed by a 52 kcal per week for 6 weeks.

Each of the members in the 2 Wk drug group 514 is subjected to an administration of the candidate intervention for a specified duration of time. Each member of the 2 Wk-drug group 514 is subjected to the administration of the candidate intervention for 2 weeks. Similarly, each member of the 4 Wk-drug group 516 is subjected to an administration of the candidate intervention for a duration of 4 weeks. Each member of the 8 Wk-drug group 518 is subjected to an administration of the candidate intervention for a duration of 8 weeks. The number of calories in the control diets fed to these groups is maintained at the normal level, e.g., 93 kcal per week for the mammalian species used in this case. The dosage of the intervention can be an effective dosage or a testing dosage. For instance, a candidate intervention can be Metformin, which may be administered in the diet of the members of the drug groups with a dosage of approximately between 0.2 mg and 2.0 gm of Metformin per kg body weight per day. In one embodiment, the 2100 mg of Metformin are added to 1 kg of the control diet. It is to be appreciated that Metformin is not the only candidate intervention. Examples of other possible candidate interventions include glucose regulatory agents such as Glipizide, and Rosiglitazone as well as countless others which may be screened as possible CR mimetics or other types of candidate intervention which may reproduce or mimic at least some of the benefits of CR.

The results of biochemical measurements (e.g., gene expression levels) from the LT-CON continuation group 506, CR2 group 508, CR4 group 510, CR8 group 512, 2 Wk-drug group 514, 4 Wk-drug group 516, and 8 Wk-drug group 518 are compared to each other. In one embodiment, the results include the changes in gene expression profiles and/or life extension for each of the groups tested. The gene expression profiles for the mice in these test groups can be determined using the methods described above. In one embodiment, the effects of the CR2 group 508, CR4 group 510, and CR8 group 512 are obtained by comparing the results from each of the CR2 group 508, CR4 group 510, and CR8 group 512 to the results of the LT-CON group 504. The effects of the 2 Wk-drug group 514, 4 Wk-drug group 516, and 8 Wk-drug group 518 are obtained by comparing the results from each of the 2 Wk-drug group 514, 4 Wk-drug group 516, and 8 Wk-drug group 518 to the results of the LT-CON group 504. The results from each of the 2 Wk-drug group 514, 4 Wk-drug group 516, and 8 Wk-drug group 518 can also be compared to the results from the CR2 group 508, CR4 group 510, and CR8 group 512.

Administering the candidate intervention to the mammalian samples for different durations of time allows for the determination of the dynamics of the candidate intervention in reproducing the effects or some of the effects of CR. Additionally, using this approach, it can be determined whether the candidate intervention can act rapidly to bring some of the CR beneficial effects to the mammalian samples (and thus mimic at least some of the effects of CR). For example, when the gene expression profiles for mice from a particular drug group (e.g., 2 Wk-drug, 4 Wk-drug, or 8 Wk-drug group) substantially correlate with the gene expression profiles for mice from a particular CR group (CR2, CR4, CR8, or LT-CR group), the candidate is identified as a CR mimetic that reproduces at least some of the effects of CR or at least some of the effects of CR administered for a particular duration of time.

FIG. 7 illustrates that, in another embodiment, a candidate intervention is administered to individuals in a mammalian group. This embodiment is particularly helpful to determine whether the candidate intervention can be used to bring the beneficial effects of CR to old mammals. The mammalian group can be a human group, a rodent group, or any other animal group. The candidate intervention can be an intervention identified using the methods previously described. In FIG. 7, a control diet program is administered to individuals in the mammalian group (box 402). After the start of old age for the mammalian group (e.g., 20 months if the mammalian group is a mouse group), the candidate intervention is administered to some of the individuals in the mammalian group (box 404). The remaining individuals of the mammalian group are maintained on the control diet program (box 406). The results (e.g., gene expression profiles or longevity) between the individuals subjected to the candidate intervention and the individuals maintained on the control diet program can be compared to each other to determine whether a candidate intervention brings benefits to the mammals tested. Additionally, the results of the candidate intervention can be compared to a pre-recorded data of CR testing to determine whether the candidate intervention can reproduce at least some of the effects of CR and be effective in treating the mammals at an older age.

In one embodiment, the candidate intervention is added concurrently with the control diet program. Thus, the candidate intervention can be mixed or added into the control diet or administered in addition to the diet. The control diet includes a normal number of calories for the particular mammals, for instance, when the mammals are mice, the number of calories of control food to be fed to each mouse may be about 93 kcal/week.

In one embodiment, to determine the effects of the candidate intervention, the gene expression profiles obtained from the mammals that were subjected to the candidate intervention are compared to the gene expression profiles obtained from the mammals that were subjected to the control diet program without the candidate intervention and to mammals that were subjected to a CR diet program. In one embodiment, a plurality of gene expression levels from the mammal subjected to the candidate intervention is compared the same type of plurality of gene expression levels from the mammals subjected to the control diet program and to mammals that were subjected to a CR diet program. The extent to which the effects (e.g., gene expression levels) of the candidate intervention match or correlate with the effects of CR will determine the likelihood that the candidate intervention is a CR mimetic. The higher the match or correlation in effects then the more likely that the candidate intervention is a CR mimetic and may be capable of reproducing at least some of the benefits of CR. The extent to which the dynamics of the effects (e.g., early responders versus late responders, etc.,) of the candidate intervention match the dynamics of the effects of CR will also determine the likelihood that the candidate intervention is a CR mimetic and thus may produce some of the benefits of CR.

In another embodiment, another group of mammals (same type of mammals) is subjected to a CR diet program (e.g., a ST-CR or a LT-CR diet program) (not shown in FIG. 7). This group of mammals may consist of old mammals, young mammals, or middle-age mammals. The results from the mammal group that was subjected to the candidate intervention can be compared to the results from the mammal group that was subjected to the CR diet program. When the gene expression levels of the mammal group that was subjected to the candidate intervention matches or substantially correlates with the corresponding gene expression levels of the mammal group that was subjected to the CR diet program, the candidate intervention can be identified as a CR mimetic or an intervention that deserves further screening. In one embodiment, the gene expression levels substantially correlate when the gene expression levels have the same direction of expression or changes and about the same magnitude of expression or changes.

It is to be noted that measuring gene expression levels is not the only method that can be used to determine the effects of the candidate intervention. Other methods that can be used include methods that measure specific protein activity levels, specific protein level changes, specific carbohydrate level changes, specific lipid level changes, specific nucleic acid levels, and the like.

It is also to be noted that animals may not be the only testing sample that can be used. Other organisms can also be used without deviating from the scope of these embodiments. For instance, a biological sample including non-mammalian organisms such as insects, nematodes, yeast, bacteria, and other organisms can also be used. In some situations, techniques may be performed in these non-mammal organisms and then candidate drugs, discovered in those organisms, can be tested in mammals (e.g., humans, mice, and monkeys).

The embodiments below take advantage of the finding that CR is rapidly reversible as discussed in several embodiments above. FIG. 8 illustrates that in one embodiment, an LT-CR diet program is administered to individuals in a mammalian group, (box 302). The mammalian group can be a human group, a rodent group, or any other animal test group. After a predetermined amount of time, e.g., 20 months in the case of mice, some individuals of the mammalian group are switched to an ST-CON diet program for a short amount of time, e.g., 2 months in the case of mice. Also after this predetermined amount of time, the remaining individuals of the mammalian group are maintained on the LT-CR for the same short amount of time, e.g., 2 months in the case of mice.

The results from this embodiment indicate that CR is reversible. This embodiment can also be used to analyze whether a candidate intervention is reversible as CR is. An example of this method of testing the reversibility of a candidate intervention is shown in FIG. 9. For example, a candidate intervention can be administered to another group of mammals (same type of mammals) for a long (or short) duration of time (as in box 302). Thus, instead of being subjected to an LT-CR as indicated in box 302, this other group of mammals is subjected to the administration of the intervention. Following that administration, the intervention is withdrawn, and the effects of the withdrawal of the intervention are compared to the effects of the withdrawal of CR. For instance, when the individuals are switched to the control diet, the reversible effects that are caused by CR should be reversed.

In one embodiment, at least one biochemical measurement (e.g., gene expression level measurement) is performed after the drug groups were exposed to the candidate intervention. The biochemical measurement is designed to show whether the candidate intervention substantially mimics or mimics at least some of the effects of CR (e.g., gene expression levels of genes known to change due to CR are measured after the candidate intervention). In one embodiment, the gene expression levels of the mammals from the particular drug group (e.g., 2 Wk-drug group, 4 Wk-drug group, and 8 Wk-drug group) are compared to the corresponding gene expression levels of the mammals from the particular CR group (CR2 group, CR4 group, or CR8group). In another embodiment, the gene expression levels for the mammals from the particular drug group are compared to the corresponding gene expression levels for the mammals from a LT-CR group (e.g., the LT-CR continuation group 118 of FIG. 3).

Continuing with FIG. 9, in one embodiment, the candidate intervention is withdrawn from a test group. In this embodiment, the administration of the candidate intervention to a mammalian group, (e.g., as in the 8 Wk-drug group 518), is withdrawn from this group. In one embodiment, the 8 Wk-drug group 518 is converted to a drug-withdrawal group and is subjected to only a control diet for a duration of time, e.g., 1-2 weeks. Similarly, a CR group can also be withdrawn from the CR diet program. In one embodiment, the CR8 group 512 is withdrawn from the CR diet program for the same duration of time (e.g., 1-2 weeks). The effects of withdrawing the candidate intervention can be compared to the effects of withdrawing the CR diet program to determine whether the intervention substantially mimics or mimics at least some of the effects of withdrawing the CR diet program. This embodiment enables one to determine whether the effects produced by a candidate intervention are substantially the same as the effects produced by the CR diet program.

In another embodiment, the CR diet program may be administered to a group for more than 8 weeks. Thus, for the CR8 group 512, instead of being subjected to the CR diet program for 8 weeks, the mammalian sample group may be subjected to an LT-CR diet program, a CR diet program with a duration longer than 8 weeks, e.g., 20 weeks in a case of mice. Similarly, the candidate intervention may be administered to a drug group for more than 8 weeks. Thus, for the 8 Wk-drug group, instead of being subjected to an administration of the candidate intervention for only 8 weeks, the mammalian sample group is subjected to the administration of the candidate intervention for longer 8 weeks, e.g., 20 weeks in a case of mice. Following the long duration, these groups may be switched to a control diet program. The results of the switch can be determined to see if the effects of the exposure and the withdrawal of the candidate intervention are similar to the effects of the exposure to and then withdrawal of the CR diet program. Normally, in one exemplary embodiment, these effects are measured by comparing gene expression levels, of genes known to change due to the introduction and/or withdrawal of CR, of members of a CR group (exposed to CR and then withdrawn from CR) to gene expression levels (for the same genes) of members of a drug group (exposed to a candidate intervention and then withdrawn from the candidate intervention).

While particular embodiments of the present invention have been shown and described, it will be obvious to those skilled in the art that changes and modifications can be made without departing from this invention in its broader aspects and, therefore, the appended claims are to encompass within their scope all such changes and modifications as fall within the scope of this invention.

1. A method of evaluating the dynamics of caloric restriction (CR) comprising:
obtaining control data from an administering of a long-term control (LT-CON) diet program;
subjecting each of several mammalian sample groups to a CR diet program wherein each of said several mammalian sample groups is subjected to said CR diet program for a different amount of time; and
comparing effects of said CR diet program between each of said several mammalian sample groups and said control data and comparing effects among members of said several mammalian sample groups.

2. The method as in claim 1 wherein said comparing effects further comprises utilizing a microarray analysis to analyze changes in each of said several mammalian sample groups that are caused by said CR diet program.

3. The method as in claim 1 wherein said comparing effects further comprises utilizing a microarray analysis to analyze changes in each of said several mammalian sample groups that are caused by said CR diet program and wherein said method further comprises validating said effects using real time RT-PCR (Reverse Transcriptase-Polymerase Chain Reaction).

4. The method as in claim 1 wherein said mammalian sample groups include one of rodents, humans, and monkeys.

5. The method as in claim 1 wherein said different amount of time varies from about a few weeks to about a few months.

6. The method as in claim 1 wherein said subjecting each of several mammalian sample groups to a CR diet program further comprises subjecting a first sample group to a CR diet program for a first predetermined period, subjecting a second sample group to said CR diet program for a second predetermined period, and subjecting a third sample group to said CR diet program for a third predetermined period, said first predetermined period being shorter than said second predetermined period and said second predetermined period being shorter than said third predetermined period.

7. The method as in claim 6 wherein said first predetermined period is about 2 weeks.

8. The method as in claim 6 wherein said second predetermined period is about 4 weeks.

9. The method as in claim 6 wherein said third predetermined period is about 8 weeks.

10. The method as in claim 1 wherein said comparing effects further comprises at least one of comparing said effects among said first sample group, said second sample group, and said third sample group to said control data and comparing said effects among said first sample group, said second sample group, and said third sample group to each other.

11. The method as in claim 1 wherein said effects including at least one of changes in gene expression levels, changes in protein levels, changes in protein activity levels, changes in carbohydrate or lipid levels, changes in nucleic acid levels, changes in rate of protein or nucleic acid synthesis, changes in protein or nucleic acid stability, changes in protein or nucleic acid accumulation levels, changes in protein or nucleic acid degradation rate, and changes in protein or nucleic acid structure or function.

12. The method as in claim 1 wherein said method further comprises fractionating genes into clusters based on how said genes are affected by said different amounts of time said CR diet program is subjected to each of said several mammalian sample groups.

13. The method as in claim 1 wherein said effects include changes in gene expression levels and wherein said method further comprises validating said changes in gene expression levels using real time RT-PCR.

14. The method as in claim 1 wherein said comparing said effects is to obtain said dynamics of CR.

15. A method of evaluating dynamics of CR comprising:
dividing a mammalian sample group into a first sample group and a second sample group;
subjecting said first sample group to a LT-CON diet program for a first predetermined period and said second sample group to a long-term caloric restriction (LT-CR) diet program for a second predetermined period;
after said first predetermined period, switching portions of said first sample group to a CR diet program for a third predetermined period;
after said second predetermined period, switching at least a portion of said second sample group to a control diet program for said third predetermined period and maintaining the other portion of said second sample group on said LT-CR diet program; and
comparing the effects of CR among members of said first sample group and said second sample group.

16. The method as in claim 15 wherein said comparing said effects is to obtain said dynamics of CR.

17. The method as in claim 15 wherein said switching portions of said first sample group to a CR diet program for different amounts of time further comprising:
switching a first portion of said first sample group to a CR diet program for a fourth predetermined period;
switching a second portion of said first sample group to said CR diet program for a fifth predetermined period;
switching a third portion of said first sample group to a CR diet program for a sixth predetermined period; and
maintaining a fourth portion of said first sample group on said LT-CON diet program.

18. The method as in claim 15 wherein said comparing effects further comprises utilizing a microarray analysis to analyze changes in each of said first sample group and said second sample group that are caused by said CR diet program at different amounts of time.

19. The method as in claim 15 wherein said comparing effects further comprises utilizing a microarray analysis to analyze changes in each of said several mammalian sample groups that are caused by said CR diet program and wherein said method further comprises validating said effects using real time RT-PCR.

20. The method as in claim 15 wherein said mammalian sample groups including one of rodents, humans, and monkeys.

21. The method as in claim 15 wherein said different amounts of time varies from a few weeks to a few months.

22. The method as in claim 15 wherein said first predetermined amount of time is 116 weeks.

23. The method as in claim 15 wherein said second predetermined amount of time is about 116 weeks.

24. The method as in claim 15 wherein said third predetermined amount of time is about 8 weeks.

25. The method as in claim 17 wherein said fourth predetermined amount of time is about 2 weeks.

26. The method as in claim 17 wherein said fifth predetermined amount of time is about 4 weeks.

27. The method as in claim 17 wherein said sixth predetermined amount of time is about 8 weeks.

28. The method as in claim 15 wherein said effects including at least one of changes in gene expression levels, changes in protein levels, changes in protein activity levels, changes in carbohydrate or lipid levels, changes in nucleic acid levels, changes in rate of protein or nucleic acid synthesis, changes in protein or nucleic acid stability, changes in protein or nucleic acid accumulation levels, changes in protein or nucleic acid degradation rate, and changes in protein or nucleic acid structure or function.

29. The method as in claim 15 wherein said method further comprises fractionating genes into clusters based on how said genes are affected by said different amounts of time said CR diet program and by said switching portions of said first sample group to a CR diet program for different amount of times and said switching at least a portion of said second sample group to a control diet program for a third predetermined period and maintaining the other portion of said second sample group on said LT-CR diet programs.

30. A method of reversing effects of CR comprising:
administering a control diet program to a mammalian sample group that has been subjected to a LT-CR diet program wherein said control diet program includes higher calorie allowance for said mammalian sample group than the calories for said LT-CR diet program.

31. The method as in claim 30 wherein said mammalian sample group is subjected to said LT-CR diet program for about 116 weeks and wherein said control diet program is administered to said mammalian sample group for about 8 weeks.

32. A method of extending longevity in an old mammal comprising:
administering a CR diet program to said old mammal.

33. The method as in claim 32 wherein said old mammal includes an old mouse.

34. The method as in claim 32 wherein said old mouse is about more than 18 months old.

35. The method as in claim 32 wherein old mammal is an old human of about more than 50 years old.

36. The method as in claim 32 wherein said administering comprises switching said old mammal to said CR diet program in at least one stage with a gradual decrease in the number of calories in diet programs.

37. A method of identifying an intervention for use in old age subjects comprising:
administering a control diet program to individuals in a first sample group;
administering, after a start of old age, at least one candidate intervention to said individuals in said first sample group; and
comparing effects of said candidate intervention to effects from a calorically restricted diet program on a second sample group.

38. The method as in claim 37 wherein said candidate intervention is, after said start of old age, administered concurrently with said control diet program.

39. The method as in claim 38 wherein said control diet program comprises a substantially normal number of calories.

40. The method as in claim 37 wherein said comparing effects comprises comparing at least one gene expression level in said individuals after said administering of said candidate intervention to at least one gene expression level from said second sample group.

41. The method as in claim 40 wherein said comparing effects comprises comparing a first plurality of gene expression levels, for a first plurality of genes, in said individuals after said administering of said candidate intervention to a second plurality of gene expression levels, for said first plurality of genes, from said second sample group and wherein said candidate intervention is identified as one deserving further screening if said first plurality of gene expression levels substantially correlates to said second plurality of gene expression levels.

42. A method as in claim 41 wherein said first plurality of gene expression levels substantially correlates to said second plurality of gene expression levels when directions of changes in gene expression levels and magnitude of changes in gene expression levels substantially match.

43. A method as in claim 37 wherein said CR diet program on said second sample group is a long-term CR diet program.

44. A method as in claim 37 wherein said CR diet program on said second sample group is a short-term CR diet program.

45. A method as in claim 37 wherein said CR diet program on said second sample group is a CR diet program for said second sample group which begins after a start of old age for said second sample group.

46. A method as in claim 37 wherein said comparing effects comprises comparing at least one biochemical measurement from said individuals after said administering of said candidate intervention to at least one biochemical measurement from said second sample group.

47. A method as in claim 46 wherein said at least one biochemical measurement comprises one of (a) a nucleic acid measurement; (b) a protein measurement; (c) a lipid measurement; and (c) a carbohydrate measurement.

48. A method of identifying an intervention comprising:
exposing a biological sample to at least one intervention;
performing at least one biochemical measurement after exposing said biological sample to said intervention, said biochemical measurement being designed to show whether said intervention mimics at least some of the effects of CR (caloric restriction);
withdrawing said intervention from said biological sample; and
performing at least one further biochemical measurement after withdrawing said intervention, said at least one further biochemical measurement being designed to show whether said withdrawing mimics at least some of the effects of withdrawing CR.

49. A method as in claim 48 wherein said performing of said at least one further biochemical measurement is after at least about 1 week after said withdrawing.

50. A method as in claim 48 wherein said intervention is administered concurrently with a control diet program having a substantially normal amount of calories.

51. A method as in claim 48 wherein said performing at least one biochemical measurement comprises comparing at least one gene expression level of said biological sample to at least one gene expression level from a calorically restricted control sample.

52. A method as in claim 51 wherein said calorically restricted control sample is subjected to a long-term CR dietary program.

53. A method as in claim 51 wherein said calorically restricted control sample is subjected to a short-term CR dietary program.

54. A method as in claim 48 wherein said performing at least one biochemical measurement comprises comparing a first plurality of gene expression levels of said biological sample, for a first plurality of genes, to a second plurality of gene expression levels from a calorically restricted control sample.

55. A method as in claim 54 wherein said performing at least one biochemical measurement comprises comparing a third plurality of gene expression levels of said biological sample, for said first plurality of genes, to a fourth plurality of gene expression levels from a sample having been withdrawn from a CR diet.

56. A method as in claim 55 wherein said intervention is identified as one deserving further screening if said first plurality of gene expression levels substantially correlates to said second plurality of gene expression levels and said third plurality of gene expression levels substantially correlates to said fourth plurality of gene expression levels.

57. A method of verifying at least some caloric restriction (CR) effects comprising:
administering a LT-CR diet program to a mammalian sample group for a predetermined amount of time;
administering a short-term control (ST-CON) diet program to some individuals of said mammalian sample group for another predetermined amount of time; and
comparing effects of each of said ST-CON diet program and said LT-CR diet program to control data from an administering of a control diet program to a second mammalian sample group and to each other.
